(19) United States
(12) Patent Application Publication
(10) Pub. No.: US 2019/0133998 A1
(43) Pub. Date: May 9, 2019

(54) TREATMENT OF TUMORS WITH INHIBITORS OF CXCL12 SIGNALING AND SUBTHERAPEUTIC AMOUNTS OF CHEMOTHERAPEUTIC AGENTS

(71) Applicant: THE GENERAL HOSPITAL CORPORATION, Boston, MA (US)

(72) Inventors: Mark C. Poznansky, Newton Center, MA (US); Patrick Reeves, Cambridge, MA (US)

(21) Appl. No.: 16/096,995

(22) PCT Filed: Apr. 26, 2017
(86) PCT No.: PCT/US2017/029582

§ 371 Date: Oct. 26, 2018

Related U.S. Application Data
(60) Provisional application No. 62/327,958, filed on Apr. 26, 2016.

(51) Int. Cl. A61K 31/395; A61K 31/337; A61P 35/00; A61K 47/64

(52) U.S. Cl. CPC A61K 31/395; A61K 47/643; A61P 35/00; A61K 31/337

(57) ABSTRACT

The invention described herein relates to methods for treating cancer in a subject by administering an effective amount of a CXCL12 signaling inhibitor and a subtherapeutic amount of an anti-cancer agent, e.g., a chemotherapeutic agent.

### STATEMENT OF PRIORITY

This application claims the benefit of U.S. Provisional Application Ser. No. 62/327,958, filed Apr. 26, 2016, the entire contents of which is incorporated by reference herein.

### FIELD OF THE INVENTION

This invention relates to methods for treating cancer in a subject by administering an effective amount of a CXCL12 signaling inhibitor and a subtherapeutic amount of an anti-cancer agent, e.g., a chemotherapeutic agent.

### BACKGROUND OF THE INVENTION

Cell movement in response to specific stimuli is observed to occur in prokaryotes and eukaryotes. Cell movement seen in these organisms has been classified into three types: chemotaxis or the movement of cells along a gradient towards an increasing concentration of a chemical; negative chemotaxis which has been defined as the movement down a gradient of a chemical stimulus; and chemokinesis or the increased random movement of cells induced by a chemical agent.

Chemotaxis and chemokinesis have been observed to occur in mammalian cells in response to the class of proteins, called chemokines. Additionally, chemorepellent activity has been observed in mammalian cells. For example, some tumor cells secrete concentrations of chemokines that are sufficient to repel immune cells from the site of a tumor, thereby reducing the immune system's ability to target and eradicate the tumor. Metastasizing cancer cells may use a similar mechanism to evade the immune system.

Agents have been described that inhibit the chemorepellent activity of tumor cells and allow the patient's immune system to target the tumor (see U.S. Patent Application Publication No. 2008/0300165, incorporated herein by reference in its entirety). However, treatment with such agents may not be sufficient to eradicate a tumor in all patients, depending on the type of tumor, size of tumor, number of metastases, site(s) of metastasis, patient's health, etc.

There remains a need for treatments and compositions that target tumors to efficiently kill tumors and/or metastasizing cancer cells.

### SUMMARY OF THE INVENTION

The CXCR4/CXCL12 and CXCR7/CXCL12 chemokine receptor/chemokine axes have been shown to be involved in the pathogenesis of a number of hematological and solid malignancies. AMD3100 is a highly specific bicyclam-based CXCR4 chemokine receptor antagonist which was originally developed as an anti-HIV medication. After it failed as a single agent to impact HIV disease it was successfully repurposed and FDA approved for use as a stem cell mobilizing agent in the context of bone marrow transplantation. AMD3100 (Plerixafor) has been used extensively for this purpose in conjunction with G-CSF and has an excellent safety profile.

Studies have shown that human epithelial ovarian cancer both secretes high levels of the chemokine, CXCL12 and expresses its cognate receptor, CXCR4. In addition, CXCR4 expression by ovarian cancer has been shown to correlate with tumor progression in humans. The CXCR4/CXCL12 axis has also been shown in vitro and in viva to play multimodal pro-tumor survival, invasion, angiogenesis and immune evasion roles. Definitive preclinical studies have been performed which demonstrate that pharmacological blockade of CXCR4 by AMD3100 elicits multi modal anti-tumor effects including anti-angiogenesis and pro-apoptotic effects as well as immune modulatory effects including the selective depletion of Tregs and increase of potent anti-tumor specific T cells in the intratumoral environment in a murine model of epithelial ovarian cancer (Righi et al., Cancer Research 2011 Aug. 15; 71(16):5522-34).

This invention relates to the treatment of a tumor that expresses a chemokine in sufficient amounts to produce a chemorepellent effect with an agent that inhibits the chemorepellent effect (e.g., an inhibitor of CXCL12 signaling) in combination with a subtherapeutic amount of one or more chemotherapeutic agents. It is contemplated the methods of this invention will be useful in the treatment of patients having a cancer, e.g., ovarian cancer, epithelial ovarian cancer, primary peritoneal cancer, fallopian tube cancer, brain cancer. The majority of patients with epithelial ovarian cancer progress to recurrent platinum resistant disease for which there are very limited therapeutic options. Ovarian cancer that has responded to initial chemotherapy but demonstrates recurrence within a relatively short period of time following the completion of treatment is considered “resistant ovarian cancer”, and the Gynocologic Oncology Group has decided that patients with documented recurrence within six months of completing initial therapy should be considered “platinum-resistant.” (Armstrong et al., Intraperitoneal cisplatin and paclitaxel in ovarian cancer, N Engl J Med. 2006 Jan. 5; 354(1):34-43).

It is contemplated that the combination of an effective amount of a CXCL12 signaling inhibitor with a subtherapeutic amount of a chemotherapeutic agent will increase the effectiveness of the chemotherapeutic agent in killing tumor cells, particularly ovarian tumor cells that have progressed to a platinum resistant status compared to the same amount of the chemotherapeutic agent when used alone.

In some embodiments of this invention, a single CXCL12 signaling inhibitor, e.g., AMD3100, or a CXCL12 signaling inhibitor, e.g., AMD3100, in combination with a subtherapeutic amount of a chemotherapeutic agent, e.g., paclitaxel (e.g., TAXOL®), may be administered to subjects having a tumor that expresses a chemokine in sufficient amounts to produce a chemorepellent effect. The subject may be, e.g., a subject having an ovarian cancer that expresses a chemokine in sufficient amounts to produce a chemorepellent effect and/or has also progressed to recurrent platinum resistant disease. In an embodiment of the invention, the CXCL12 signaling inhibitor may be administered to a subject in need thereof in an amount that enhances the penetration of immune cells into a tumor and the chemotherapeutic agent may be administered in a subtherapeutic amount at the same time, before or after administration of the CXCL12 signaling inhibitor.

However, in some embodiments, the chemotherapeutic agent is not administered before the CXCL12 signaling inhibitor.

An embodiment of this invention is a method for killing a cancer cell expressing an amount of a chemokine sufficient to produce a chemorepellent effect in a subject in need thereof, which method comprises:

a) contacting the cancer cell with an amount of a CXCL12 signaling inhibitorfor a sufficient period of time to inhibit the chemorepellent effect and to increase migration of immune cells to the cancer cell;
b) contacting the cancer cell with a subtherapeutic amount of a chemotherapeutic agent, and
c) optionally repeating steps a) and b) as necessary to kill the cancer cell.

In some embodiments, contacting of the cancer cell with the CXCL12 signaling inhibitor may be periodic.

In an embodiment of the methods of this invention, the CXCL12 signaling inhibitor and chemotherapeutic agent may be administered at the same time/concurrently or sequentially. “In combination” refers to any combination, including sequential or simultaneous administration. In one embodiment, the CXCL12 signaling inhibitor may be administered separately from the chemotherapeutic agent.

In some embodiments of the methods of this invention, the CXCL12 signaling inhibitor may be administered before administering the chemotherapeutic agent.

In some embodiments of the methods of this invention, the CXCL12 signaling inhibitor may be administered concurrently or up to about 3 days to about 10 days before administering the chemotherapeutic agent.

In some embodiments, the CXCL12 signaling inhibitor may be administered in a continuous manner for a defined period. In another embodiment, the CXCL12 signaling inhibitor may be administered in a pulsatile manner. For example, the CXCL12 signaling inhibitor may be administered intermittently over a period of time.

In an embodiment, the CXCL12 signaling inhibitor and anti-cancer agent(s), (e.g., chemotherapeutic agents, e.g., a taxane, a paclitaxel including TAXOL® and ABRAXANE®), may be administered sequentially. For example, the CXCL12 signaling inhibitor may be administered for a period of time sufficient to reduce or attenuate the chemotherapeutic effect of the tumor, e.g., such that the CXCL12 signaling inhibitor inhibits CXCL12 signaling; the anti-cancer agent, e.g., chemotherapeutic such as paclitaxel (e.g., TAXOL®, ABRAXANE®), may then be administered for a period of time during which the chemorepellent effect of the tumor is reduced or attenuated. In some embodiments, the CXCL12 signaling inhibitor and chemotherapeutic agent may be administered sequentially in an alternating manner.

In some embodiments, the CXCL12 signaling inhibitor and/or chemotherapeutic agent may be administered intravenously, subcutaneously, orally, or intraperitoneally. As above, such administration may occur in any order provided that the timing of such administration provides a desired endpoint.

A CXCL12 signaling inhibitor may be any such inhibitor now known or later identified that acts to reduce the chemorepellent or CXCL12 signaling of a cancer cell. CXCL12 signaling inhibitors are known in the art, and include, but are not limited to, AMD3100 (mozobil/plerixafor), AMD11070 (also called AMD070), AMD12118, AMD11814, AMD13073, FAMD3465, C131, BKT140, CTCE-9908, KRH-1636, KRH-2731, KRH-3955, TC14012, BMS-936564/MDX-1338, LY2510924, GSK812397, T-20, T-22, T-140, TE-14011, T-14012, TN14003, TAK-779, AK602, SCH-351125, Tannic acid, NSC 651016, thalidomide, GF 109230X, and an antibody that interferes with ability of a cancer cell to act in a chemorepellent manner (e.g., interferes with CXCL12 signaling).

The chemokine that is expressed by the cancer cells in an amount sufficient to produce a chemorepellent effect (e.g., repel immune cells from a tumor) includes, but is not limited to, e.g., CXCL12 or interleukin 8.

In an embodiment of this invention, the cancer cell is a solid tumor cell, an ovarian cancer cell, e.g., an epithelial ovarian cancer cell, a fallopian tube cancer cell, or a primary peritoneal cancer cell. In some embodiments, the ovarian cancer cell may be e.g., a cancer cell that has progressed to platinum resistance.

Also an embodiment of this invention is a method for killing a cancer cell in a solid tumor expressing a chemokine at a concentration sufficient to produce a chemorepellent effect in a subject in need thereof, which method comprises, a) administering an effective amount of a CXCL12 signaling inhibitor into the tumor for a sufficient time to increase penetration of immune cells into the tumor; and b) subsequently administering a subtherapeutic amount of a chemotherapeutic agent to the subject, thereby killing the cancer cell in the solid tumor. In some embodiments, the CXCL12 signaling inhibitor may be administered directly into the tumor.

An additional embodiment of this invention is a method for treating a tumor expressing a chemokine at a concentration sufficient to produce a chemorepellent effect in a subject in need thereof, which method comprises: a) injecting or infusing an amount of a CXCL12 signaling inhibitor into said tumor for a sufficient time to increase penetration of immune cells into the tumor; and b) subsequently administering subtherapeutic amount of the chemotherapeutic agent to the subject, thereby treating the tumor. In some embodiments, the CXCL12 signaling inhibitor may be injected or infused directly into the tumor.

Another embodiment of this invention is a method for enhancing the therapeutic effect of a chemotherapeutic agent on a tumor expressing an amount of a chemokine sufficient to produce a chemorepellent effect, which method comprises, a) administering an effective amount of a CXCL12 signaling inhibitor to a subject having the tumor for a sufficient time to increase penetration of immune cells into the tumor; and b) administering a subtherapeutic amount of the chemotherapeutic agent to the subject, wherein the therapeutic effect of the subtherapeutic amount of the chemotherapeutic agent on the tumor is enhanced as compared to the effect on the tumor of the subtherapeutic amount of the chemotherapeutic agent administered without the CXCL12 signaling inhibitor.

Another embodiment of this invention is a method for enhancing the therapeutic effect of a chemotherapeutic agent on a tumor expressing an amount of a chemokine sufficient to produce a chemorepellent effect, which method comprises, a) selecting a subject having a tumor expressing an amount of a chemokine sufficient to produce a chemorepellent effect, b) administering an effective amount of a CXCL12 signaling inhibitor to the subject for a sufficient time to increase penetration of immune cells into the tumor; and c) administering a subtherapeutic amount of a chemotherapeutic agent to the subject, wherein the therapeutic effectiveness of the subtherapeutic amount of the chemotherapeutic agent is enhanced as compared to the subtherapeutic amount administered without the CXCL12 signaling inhibitor.

An embodiment of this invention is a method for increasing immune cell migration into a tumor and the effectiveness of the chemotherapeutic agent, which method comprises
a) identifying a tumor having a chemorepellent property whereby immune cells are repelled from the tumor,
b) contacting the tumor with an amount of a CXCL12 signaling inhibitor for a sufficient period of time to inhibit the chemorepellent effect;
c) contacting the tumor with a subtherapeutic amount of a chemotherapeutic agent, and
d) optionally repeating steps b) and c),
   whereupon the migration of immune cells into the tumor is increased, thereby increasing the effectiveness of the chemotherapeutic agent.
   In some embodiments, contacting of the cancer cell with the CXCL12 signaling inhibitor may be periodic.

In some embodiments, a subject having a tumor expressing an amount of chemokine sufficient to produce a chemorepellent effect or a tumor having a chemorepellent property whereby immune cells are repelled from the tumor may be selected or identified using immune histochemistry, western blotting and/or ELISA assay. Tumor cells express more chemokine (e.g., CXCL12) than equivalent normal epithelial or other normal matched healthy tissues and this higher level of expression can be detected using immune histochemistry, western blotting and ELISA assay.

In embodiments of this invention, the CXCL12 signaling inhibitor and chemotherapeutic agent may be administered to a subject who has a tumor which expresses a chemokine in an amount sufficient to produce a chemorepellent effect. In embodiments of this invention such tumor may be a solid tumor. In embodiments of this invention the tumor may be an ovarian tumor. In some embodiments, the subject may be a subject who has or has had a recurrence of epithelial ovarian cancer, primary peritoneal cancer, or fallopian tube cancer.

In an embodiment of this invention, the chemotherapeutic agent may be a taxane.

In an embodiment of this invention, the chemotherapeutic agent may be a paclitaxel. Paclitaxel as recited herein includes any formulation containing a paclitaxel, such as TAXOL® and ABRAXANE®. Paclitaxel compounds are well known in the art.

It is contemplated that the therapeutic effect, e.g., inhibition of the proliferation or growth of tumor cells and/or killing of tumor cells, achieved by the co-administration of CXCL12 signaling inhibitor and a subtherapeutic amount a chemotherapeutic agent will be synergistic, i.e., the combination of the chemotherapeutic agent with the CXCL12 signaling inhibitor will achieve an effect that is greater than the sum of the effect achieved by either the chemotherapeutic agent alone or the CXCL12 signaling inhibitor alone. For example, the combined effect of the use of a CXCL12 signaling inhibitor with a chemotherapeutic agent may increase the effectiveness of the chemotherapeutic agent by at least about 5% to about 100% or more (e.g., about 5, 10, 15, 20, 25, 30, 35, 40, 45, 50, 55, 60, 65, 70, 75, 80, 85, 90, 95, 100, 125, 150, 200% or more, and any range or value therein).

### BRIEF DESCRIPTION OF THE DRAWINGS

FIGS. 1A-1F show CXCL12, CXCR4, and CXCR7 expression on ID8 and TOV-112D cells. FIGS. 1A-1C show ID8 cells treated with AMD3100 (10 μM). FIGS. 1D-1F show TOV-112D cells treated with AMD3100 (5 μM). Cells were treated either overnight or for 4 hrs prior to harvest either overnight or for 4 hrs prior to harvest. Representative histogram plots for CXCR4, CXCR7, and CXCL12 presented next to bar graphs. Bars are representative of data from two independent experiments performed in duplicate normalized to mean fluorescence intensity set of untreated controls set to 100% and negative control set 0%. One-way Anova with Bonferroni correction was conducted to determine significance as compared to untreated control A-C. ID8 CXCL12, CXCR4, and CXCR7 expression respectively. (* p<0.0332, **p<0.0021, ***p<0.0002). D-F. TOV-112D CXCL12, CXCR4, and CXCR7 expression respectively. (*p<0.0223).

FIGS. 2A-2B show the effect of AMD3100 and TAXOL treatment on ID8 and TO112D cell proliferation. FIG. 2A shows ID8 cells treated with carrier, AMD3100 10 μM, 5 nM TAXOL, or both, measured in triplicate (*p=0.0228, ****p<0.0001, from 3 experiments) FIG. 2B shows TOV-112D cells treated with AMD3100 5 μM, 1 nM TAXOL, or both, measured in triplicate (*p=0.0210, ****p<0.0001, from 3 experiments). Bars represent mean, error bars SEM, one-way ANOVA analysis with Bonferroni correction.

FIGS. 3A-3B show the titration of TAXOL effects on ID8 and TOV-112D proliferation. ID8 (A) or TOV-112D (B) cells cultured in media containing a TAXOL range from 1 μM to 5 nM and proliferation measured by CyQuant assay. Graphs represent mean of 3 wells, error bar SEM.

FIGS. 4A-4B show the effect of sequential delivery of AMD3100 and Taxol on ID8 and TOV-112D cell proliferation. ID8 and TOV-112D cells were cultured in control media or media containing AMD3100, harvested, and equal numbers of live cells plated. Re-plated cells were cultured in control media, AMD3100, Taxol, or both. Bars represent mean and SEM of 2 (FIG. 4A) or 3 (FIG. 4B) independent experiments. 2-way ANOVA with Bonferroni, Angled asteriks indicate significance relative to post-treatment control within each pre-treatment category. Pairwise statistical comparisons indicated with brackets. Non-significant comparisons not shown. FIG. 4A ID8*p=0.0028, ****p 0.0001 FIG. 4B. TOV-112D*p=0.0328, *b p=0.015, **p=0.0035, **b p=0.0081, ***p=0.0002, ***b p=0.0005, ****p<0.0001.

FIGS. 5A-5B show AMD3100 and Taxol limit ID8 and TOV-112D colony formation. FIG. 5A. ID8 (FIG. 5A) or TOV-112D (FIG. 5B) cells were allowed to form colonies in soft agar containing AMD3100 or Taxol, or both. Following incubation, colonies were imaged and quantified. Graphs represent normalized mean of 3 wells, error bar SEM, 2 independent experiments, one-way ANOVA analysis with Bonferroni correction (compared to control). **p=0.0054, **b p=0.0016, ****p<0.0001

FIG. 6 shows survival data of tumor bearing mice were treated with 1 mg/kg AMD-3100, 10 mg/kg TAXOL or saline.

FIG. 7 shows survival data of tumor bearing mice were treated with 1 mg/kg AMD-3100, 30 mg/kg ruxolitinib (ruxo) or saline.

FIGS. 8A-8B show survival data of tumor bearing mice were treated with 1 mg/kg AMD-3100, 10 mg/kg VIC-800 or saline.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

All publications, patent applications, patents, nucleotide sequences, amino acid sequences and other references mentioned herein are incorporated by reference in their entirety.

### Definitions

Unless defined otherwise, all technical and scientific teams used herein have the same meaning as commonly understood by one of ordinary skill in the art to which this invention belongs. The terminology used in the description of the invention herein is for the purpose of describing particular embodiments only and is not intended to be limiting of the invention.

In this specification and in the claims that follow, reference will be made to a number of terms that shall be defined to have the following meanings:

The terminology used herein is for the purpose of describing particular embodiments only and is not intended to be limiting of the invention. As used herein, the singular forms of “a”, “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

All numerical designations, e.g., pH, temperature, time, concentration, amounts, and molecular weight, including ranges, are approximations which are varied (+) or (−) by 20%, 15%, 10%, 5%, 1%, 0.5%, 0.1%, and the like, as appropriate. It is to be understood, although not always explicitly stated, that all numerical designations may be preceded by the term “about.” It is also to be understood, although not always explicitly stated, that the reagents described herein are merely exemplary and that equivalents of such are known in the art.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Optional” or “optionally” means that the subsequently described event or circumstance can or cannot occur, and that the description includes instances where the event or circumstance occurs and instances where it does not.

The term “comprising” or “comprises” is intended to mean that the compositions and methods include the recited elements, but not excluding others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of other ingredients and substantial method steps recited. Embodiments defined by each of these transition terms are within the scope of this invention.

The terms “patient,” “subject,” “individual,” and the like are used interchangeably herein, and refer to any animal, or cells thereof whether in vitro or in situ, amenable to the methods described herein. In some embodiments, the patient, subject, or individual may be a mammal. In some embodiments, the mammal may be a mouse, a rat, a guinea pig, a non-human primate, a dog, a cat, or a domesticated animal (e.g. horse, cow, pig, goat, sheep). In representative embodiments, the patient, subject or individual may be a human.

The term “treating” or “treatment” covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder (i.e., delay in progression or onset); and/or (iv) inhibiting, relieving, or slowing progression of one or more symptoms of the disease or disorder. For example, treatment of a cancer or tumor includes, but is not limited to, reduction in size of the tumor, elimination of the tumor and/or metastases thereof, remission of the cancer, inhibition of metastasis of the tumor, reduction or elimination of at least one symptom of the cancer, and the like.

The term “administering” or “administration” of an inhibitor, agent, drug, or a natural killer cell to a subject includes any route of introducing or delivering to a subject a compound to perform its intended function. Administration can be carried out by any suitable route, including, but not limited to, orally, intranasally, parenterally (intravenously, intramuscularly, intraperitoneally, or subcutaneously), and/or topically. Administration includes self-administration and the administration by another.

It is also to be appreciated that the various modes of treatment or prevention of medical diseases and conditions as described are intended to mean “substantial,” which includes total but also less than total treatment or prevention, and wherein some biologically or medically relevant result is achieved.

The term “separate” administration refers to an administration of at least two active ingredients at the same time or substantially the same time by different routes or by separate routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients and in that case there is no simultaneous treatment.

The term “concurrent” or “simultaneous” therapeutic use refers to the administration of at least two active ingredients at the same time or at substantially the same time, typically within plus or minus 12 hours of each other. In some embodiments, the at least two active ingredients may be in the same composition or in different compositions. In some embodiments, the at least two active ingredients may be delivered by the same route or by different routes.

As used herein, the tell “enhance” or “increase” refers to an increase in the specified parameter of at least about 10%, 15%, 25%, 50%, 75%, 100%, 150%, 200%, 300%, 400%, 500% or more as compared to a control. Thus, for example, a method for enhancing the therapeutic effect of a chemotherapeutic agent on a tumor expressing an amount of a chemokine sufficient to produce a chemorepellent effect as described herein may result in therapeutic effect of the subtherapeutic amount of the chemotherapeutic agent on the tumor that is enhanced by at least about 10%, 15%, 25%, 50%, 75%, 100%, 150%, 200%, 300%, 400%, 500% or more as compared to a control (e.g., the subtherapeutic amount administered without the CXCL12 signaling inhibitor). In another example, the present invention provides a method for increasing immune cell migration into a tumor wherein the migration of the cells into the tumor is increased by least about 10%, 15%, 25%, 50%, 75%, 100%, 150%, 200%, 300%, 400%, 500% or more as compared to a control.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “therapeutic” as used herein means a treatment and/or prophylaxis. A therapeutic effect is obtained by suppression, remission, or eradication of a disease state.

The term “therapeutically effective amount” or “effective amount” refers to an amount of an agent (e.g., a chemotherapeutic agent, an inhibitor of a chemorepellent effect, e.g., aCXCL12 signaling inhibitor) that, when administered, is sufficient to provide some improvement or benefit to the subject. Alternatively stated, an “effective,” “prophylactically effective,” or “therapeutically effective” amount is an amount that will provide some delay, alleviation, mitigation, or decrease in at least one clinical symptom in the subject. Those skilled in the art will appreciate that the effects need not be complete or curative, as long as some benefit is provided to the subject. For example, an effective amount of a CXCL12 signaling inhibitor may be an amount sufficient to inhibit or reduce CXCL12 signaling in a cancer cell or tumor (e.g. to attenuate a chemorepellent effect from the tumor or cancer cell). The therapeutically effective amount of an agent will vary depending on the tumor being treated and its severity as well as the age, weight, etc., of the subject to be treated. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder.

As used herein, the term “subtherapeutic”, is used to describe an amount of a chemotherapeutic agent less than the amount conventionally used to treat a cancer. For example, a sub-therapeutic amount is an amount less than that defined by the manufacturer as being required for therapy. For example for paclitaxel, including TAXOL® and ABRAXANE®, a subtherapeutic amount may be about 10%, 20% 30%, 35%, 40%, 45%, 50%, 55%, 60%, 65%, 70%, or 75%, of the amount defined by the manufacturer as being required for therapy.

The term “kill” with respect to a cell/cell population is directed to include any type of manipulation that will lead to the death of that cell/cell population.

“Antibodies” as used herein include polyclonal, monoclonal, single chain, chimeric, humanized and human antibodies, prepared according to conventional methodology.

“Cytokine” is a generic term for non-antibody, soluble proteins which are released from one cell subpopulation and which act as intercellular mediators, for example, in the generation or regulation of an immune response. See Human Cytokines: Handbook for Basic & Clinical Research (Aggarwal, et al. eds., Blackwell Scientific, Boston, Mass. 1991) (which is hereby incorporated by reference in its entirety for all purposes).

“CXCR4/CXCL12 antagonist” refers to a compound that antagonizes CXCL12 binding to CXCR4 or otherwise reduces the chemorepellent effect of CXCL12.

“CXCR7/CXCL12 antagonist” refers to a compound that antagonizes CXCL12 binding to CXCR7 or otherwise reduces the chemorepellent effect of CXCL12.

By “chemorepellant activity” it is meant the ability of an agent to repel (or chemorepel) a eukaryotic cell with migratory capacity (i.e., a cell that can move away from a repellant stimulus). Accordingly, an agent with chemorepellant activity is a “chemorepellant agent.” Such activity can be detected using any of a variety of systems well known in the art (see, e.g., U.S. Pat. No. 5,514,555 and U.S. Patent Application Pub. No. 2008/0300165, each of which is incorporated by reference herein in its entirety). A system for use herein is described in U.S. Pat. No. 6,448,054, which is incorporated herein by reference in its entirety.

The terms “chemorepellant effect” refers to the chemorepellant effect of a chemokine secreted by a cell, e.g. a tumor cell. Usually, the chemorepellent effect is present in an area around the cell wherein the concentration of the chemokine is sufficient to provide the chemorepellent effect.

Some chemokines, including interleukin 8 and CXCL12, may exert chemorepellent activity at high concentrations (e.g., over about 10 nM), whereas lower concentrations exhibit no chemorepellent effect and may even be chemoattractant. Thus, a solid tumor expressing a chemokine at a concentration sufficient to produce a chemorepellent effect may be at a concentration of least about 10 nM to about 1 μM. In some embodiments, a concentration sufficient to produce a chemorepellent effect may be about 25 nM to about 800 nM, about 50 nM to about 600 nM, or about 100n M to 500 nM, about 100 nM to about 1 μM, and the like. In some embodiments, a concentration sufficient to produce a chemorepellent effect may be about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000 nM, and any range or value therein.

The term “anti-chemorepellent effect” refers to the effect of an agent (e.g., a CXCL12 signaling inhibitor) to attenuate or eliminate the chemorepellent effect of the chemokine. A chemorepellent effect includes, for example, the effect of increasing migration of immune cells to a cancer cell or tumor or the effect of increasing penetration of an immune cell into a tumor.

“Immune cells” as used herein are cells of hematopoietic origin that are involved in the specific recognition of antigens. Immune cells include antigen presenting cells (APCs), such as dendritic cells or macrophages, B cells, T cells, etc. In one embodiment, the immune cell is a cell that is repelled by a chemorepellent response of a tumor.

The term “anti-cancer therapy” as used herein refers to traditional cancer treatments, including chemotherapy and radiotherapy, as well as vaccine therapy.

In some aspects, the methods of this invention can be effective in treating cancers, which exhibit a chemorepellent property. Such cancers include, but are not limited to, “ovarian cancer”. It may be necessary to evaluate the subject before administering a CXCL12 signaling inhibitor and chemotherapeutic agent as described herein. Such evaluation can use assays well known in the art (e.g., transmigration assays, immunohistochemistry, western blot from tissue lysates and ELISA assays from tissue lysates).

### Agents for Inhibition of CXCL12 Signaling

Many tumors have chemorepellent effects on, e.g., immune cells, due to chemokines that are secreted by the tumor cells. High concentrations of chemokines secreted by the tumor cells can have chemorepellant effects on cells, whereas lower concentrations do not have such effects or even result in chemoattraction. For example, T-cells are repelled by CXCL12 (SDF-1) by a concentration-dependent and CXCR4 receptor-mediated mechanism. This invention is predicated on the surprising discovery that agents which inhibit CXCL12 signaling as described herein can reduce the chemorepellent effects of the tumors, thereby allowing immune cells and other anti-cancer agents to better access and kill the tumor cells. The benefits of this invention arise from the ability of a patient's immune cells to cooperate synergistically with the chemotherapeutic agent to treat the tumor.

A CXCL12 signaling inhibitor may be any such inhibitor known in the art, for example a CXCL12 signaling inhibitor as described in U.S. Patent Application Publication No. 2008/0300165, which is hereby incorporated by reference in its entirety.

A CXCL12 signaling inhibitor may include any inhibitor that interferes with ability of a chemorepellent to act in a chemorepellent manner

Certain chemokines, including IL-8 and CXCL12 can serve as chemorepellents at high concentrations (e.g., above 100 nM). Blocking the chemorepellent effect of high concentrations of a chemokine secreted by a tumor can be accomplished, for example, by an antichemorepellent agent (e.g., a CXCL12 signaling inhibitor), which can interfere with the ability of a chemorepellent agent to act in a chemorepellent manner. For example, antibodies that interfere with ability of a chemorepellent to act in a chemorepellent manner are anti-chemorepellent agents. Anti-chemorepellent agents that, e.g., reduce the amount of a chemorepellent cytokine secreted by the cells, and/or inhibit binding of a chemokine to a target receptor, are also encompassed by the present invention. Where desired, this effect can be achieved without inhibiting the chemotactic action of a monomeric chemokine.

In some embodiments, anti-chemorepellent agent can include, but is not limited to, an inhibitor of CXCL12 signaling, a CXCR4 antagonist, CXCR3 antagonist, CXCR4/CXCL12 antagonist and/or selective PKC inhibitor.

An inhibitor of CXCL12 signaling may be any molecule that inhibits the CXCL12/CXCR4/CXCR7 axis. The inhibitor may completely or partially inhibit signaling through the CXCL12/CXCR4/CXCR7 axis when administered to a subject, e.g., providing at least about 30%, 40%, 50%, 60%, 70%, 80%, 90% or more inhibition. Inhibitors may include, without limitation, molecules that inhibit expression of CXCL12 or CXCR4 or CXCR7 (e.g., antisense or siRNA molecules), molecules that bind to CXCL12 or CXCR4 or CXCR7 and inhibit their function (e.g., antibodies or aptamers), molecules that inhibit dimerization of CXCL12 or CXCR4 or CXCR7, and antagonists of CXCR4 or CXCR7. In one embodiment, the inhibitor of CXCL12 signaling is a CXCR4 antagonist. The CXCR4 antagonist can be but is not limited to AMD3100, AMD11070 (also called AMD070), AMD12118, AMD11814, AMD13073, FAMD3465, C131, BKT140, CTCE-9908, KRH-2731, TC14012, KRH-3955, BMS-936564/MDX-1338, LY2510924, GSK812397, KRH-1636, T-20, T-22, T-140, TE-14011, T-14012, or TN14003, or an antibody that interferes with the dimerization of CXCR4. In one embodiment, the CXCR4 antagonist is AMD3100 (plerixafor). AMD3100 is described in U.S. Pat. No. 5,583,131, which is incorporated by reference herein in its entirety. In one embodiment, the inhibitor of CXCL12 signaling is a CXCR7 antagonist. The CXCR7 antagonist can be but is not limited to CCX771, CCX754, or an antibody that interferes with the dimerization of CXCR7. In certain embodiments, the inhibitor of CXCL12 signaling is not an antibody. In certain embodiments, the inhibitor of CXCL12 signaling is not a heparinoid. In certain embodiments, the inhibitor of CXCL12 signaling is not a peptide

In one embodiment, example inhibitors include, but are not limited to, AMD3100, AMD11070 (i.e., AMD070), AMD12118, AMD11814, AMD13073, FAMD3465, C131, BKT140, CTCE-9908, KRH-1636, KRH-2731, KRH-3955, TC14012, BMS-936564/MDX-1338, LY2510924, GSK812397, T-20, T-22, T-140, TE-14011, T-14012, TN14003, TAK-779, AK602, SCH-351125, tannic acid, NSC 651016, thalidomide, and/or GF 109230X.

A CXCR4 antagonist can include, but is not limited to, AMD3100, KRH-1636, T-20, T-22, T-140, TE-14011, T-14012, and/or TN14003, and/or an antibody that interferes with ability of a chemorepellent agent to act in a chemorepellent manner (e.g., chemorepellant to, for example, immune cells).

A CXCR3 antagonist can include, but is not limited to, TAK-779, AK602, and/or SCH-351125, and/or an antibody that interferes with ability of a chemorepellent to act in a chemorepellent manner.

A CXCR4/CXCL12 antagonist can include but is not limited to Tannic acid, NSC 651016, and/or an antibody that interferes with ability of a chemorepellent to act in a chemorepellent manner.

A selective PKC inhibitor can include, but is not limited to, thalidomide and/or GF 109230X.

In some embodiments, an inhibitor of CXCL12 signaling may be AMD3100 (plerixafor). AMD3100 is described in U.S. Pat. No. 5,583,131, which is incorporated by reference herein in its entirety.

In some embodiments, an inhibitor of CXCL12 signaling may be coupled with a molecule that allows targeting of a tumor. In some embodiments, an inhibitor of CXCL12 signaling may be coupled with (e.g., bound to) an antibody specific for the tumor to be targeted. In some embodiments, an inhibitor of CXCL12 signaling coupled to the molecule that allows targeting of the tumor may be administered systemically.

CXCL12 expression by a tumor may also promote tumor growth, angiogenesis, and metastasis. Accordingly, methods for inhibiting tumor growth, angiogenesis, and metastasis are contemplated by this invention.

In one embodiment, an inhibitor of CXCL12 signaling may be administered in combination with an additional compound that enhances the anti-chemorepellent activity of the inhibitor. In one embodiment, the additional compound may be granulocyte colony stimulating factor (G-CSF). In one embodiment, G-CSF is not administered.

### Chemotherapy Agents

In one aspect of the present invention, an inhibitor of CXCL12 signaling may be administered in combination with a chemotherapy agent. The chemotherapy agent may be any agent having a therapeutic effect on one or more types of cancer. Many chemotherapy agents are currently known in the art. Types of chemotherapy drugs include, by way of non-limiting example, alkylating agents, antimetabolites, anti-tumor antibiotics, topoisomerase inhibitors, mitotic inhibitors, corticosteroids, and the like.

Non-limiting examples of chemotherapy drugs include: nitrogen mustards, such as mechlorethamine (nitrogen mustard), chlorambucil, cyclophosphamide (CYTOXAN®), ifosfamide, and melphalan); Nitrosoureas, such as streptozocin, carmustine (BCNU), and lomustine; alkyl sulfonates, such as busulfan; Triazines, such as dacarbazine (DTIC) and temozolomide (TEMODAR®); ethylenimines, such as thiotepa and altretamine (hexamethylmelamine); platinum drugs, such as cisplatin, carboplatin, and oxalaplatin; 5-fluorouracil (5-FU); 6-mercaptopurine (6-MP); Capecitabine (Xeloda®); Cytarabine (ARA-C®); Floxuridine; Fludarabine; Gemcitabine (Gemzar®); Hydroxyurea; Methotrexate; Pemetrexed (ALIMTA®); anthracyclines, such as Daunorubicin, Doxorubicin (ADRIAMYCIN®), Epirubicin, Idarubicin; Actinomycin-D; Bleomycin; Mitomycin-C; Mitoxantrone; Topotecan; lrinotecan (CPT-11); Etoposide (VP-16); Teniposide; Mitoxantrone; Taxanes: paclitaxel (e.g., TAXOL®, ABRAXANE®) and docetaxel (TAXOTERE®); Epothilones: ixabepilone (IXEMPRA®); Vinca alkaloids: vinblastine (VELBAN®), vincristine (ONCOVIN®), and vinorelbine (NAVELBINE®); Estramustine (EMCYT®); Prednisone; Methylprednisolone (SOLUMEDROL®); Dexamethasone (DECADRON®); L-asparaginase; and/or bortezomib (VELCADE®). Additional chemotherapy agents are listed, for example, in U.S. Patent Application Pub. No. 2008/0300165, which is incorporated herein by reference in its entirety.

Doses and administration protocols for chemotherapy drugs are well-known in the art. The skilled clinician can readily determine the proper dosing regimen to be used, based on factors including the chemotherapy agent(s) administered, type of cancer being treated, stage of the cancer, age and condition of the patient, patient size, location of the tumor, and the like. The skilled clinician can readily determine subtherapeutic amounts of such chemotherapy agents.

### Cancers

Cancers or tumors that can be treated by the compounds and methods described herein include, but are not limited to: biliary tract cancer; brain cancer, including glioblastomas and medulloblastomas; breast cancer; cervical cancer; choriocarcinoma; colon cancer; endometrial cancer; esophageal cancer, gastric cancer; hematological neoplasms, including acute lymphocytic and myelogenous leukemia; multiple myeloma; AIDS associated leukemias and adult T-cell leukemia lymphoma; intraepithelial neoplasms, including Bowen's disease and Paget's disease; liver cancer (hepatocarcinoma); lung cancer; lymphomas, including Hodgkin's disease and lymphocytic lymphomas; neuroblastomas; oral cancer, including squamous cell carcinoma; ovarian cancer, including those arising from epithelial cells and those that have progressed to platinum-resistant cancer, stromal cells, germ cells and mesenchymal cells; pancreas cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; thyroid cancer, including thyroid adenocarcinoma and medullar carcinoma; and renal cancer including adenocarcinoma and Wilms tumor. In some embodiments, cancers or tumors that may be treated by the compounds and methods of the present invention include cancers and/or tumors escaping immune recognition include glioma, colon carcinoma, colorectal cancer, lymphoid cell-derived leukemia, choriocarcinoma, upper GI cancer, head and neck cancer, cervical cancer, ovarian cancer, germ cell tumors, prostate cancer, breast cancer and/or melanoma.

In some embodiments, the tumor may be a solid tumor. In some embodiments, the tumor/cancer may be a leukemia. In further embodiments, the tumor may be a tumor that over-expresses CXCL12. In one embodiment, tumor expression of CXCL12 can be evaluated prior to administration of a composition as described herein. For example, a subject having a tumor that is determined to express or over-express CXCL12 may be treated using a method and/or composition as described herein.

In one embodiment, the tumor may be a brain tumor. It is contemplated that a brain tumor, e.g., an inoperable brain tumor, can be injected with a composition described herein. In one embodiment, an inhibitor of CXCL12 signaling may be administered directly to a brain tumor via a catheter into a blood vessel within or proximal to the brain tumor. Further discussion of catheter or microcatheter administration is described below.

### Dose and Administration

The compositions, as described herein, are administered in effective amounts. The effective amount will depend upon the mode of administration, the particular condition being treated and the desired outcome. It will also depend upon, as discussed above, the stage of the condition, the age and physical condition of the subject, the nature of concurrent therapy, if any, and like factors well known to the medical practitioner. For therapeutic applications, it is that amount sufficient to achieve a medically desirable result.

The anti-cancer agent, e.g., chemotherapeutic agent, may be administered by any appropriate method. Dosage, treatment protocol, and routes of administration for anti-cancer agents, including chemotherapeutic agents, radiotherapeutic agents, and anti-cancer vaccines, are known in the art and/or within the ability of a skilled clinician to determine, based on the type of treatment, type of cancer, etc.

It should be understood that while a subtherapeutic dose of the chemotherapeutic agent displays synergy with an inhibitor of CXCL12 signaling, one of skill in the art will immediately recognize that higher amounts, including therapeutic amounts, of the chemotherapeutic agent will also produce synergy when used in combination with an effective amount of an inhibitor of CXCL12 signaling. It is understood the benefits of this invention are bestowed by allowing immune cells to penetrate through the chemorepellent wall (e.g., increase migration of immune cells to a cancer cell, increase penetration of an immune cell into a tumor), thereby enhancing the overall therapy of the combination used herein. Such synergistic mixtures allow for less chemotherapeutic agent to be used, although higher amounts, including therapeutic amounts, of the chemotherapeutic agent are also contemplated and are contemplated to be within the scope of this invention.

In some embodiments, the dose of the CXCL12 signaling inhibitor of the present invention may be from about 5 mg/kg body weight per day to about 50 mg/kg per day, inclusive of all values and ranges therebetween, including endpoints. In one embodiment, the dose may be from about 10 mg/kg to about 50 mg/kg per day. In one embodiment, the dose may be from about 10 mg/kg to about 40 mg/kg per day. In one embodiment, the dose may be from about 10 mg/kg to about 30 mg/kg per day. In one embodiment, the dose may be from about 10 mg/kg to about 20 mg/kg per day. In one embodiment, the dose does not exceed about 50 mg per day.

In one embodiment, the dose of the CXCL12 signaling inhibitor may be from about 50 mg/kg per week to about 350 mg/kg per week, inclusive of all values and ranges therebetween, including endpoints. In one embodiment, the dose of the CXCL12 signaling inhibitor may be about 50 mg/kg per week. In one embodiment, the dose of the CXCL12 signaling inhibitor may be about 60 mg/kg per week. In one embodiment, the dose of the CXCL12 signaling inhibitor may be about 70 mg/kg per week. In one embodiment, the dose of the CXCL12 signaling inhibitor may be about 80 mg/kg per week. In one embodiment, the dose of the CXCL12 signaling inhibitor may be about 90 mg/kg per week. In one embodiment, the dose of the CXCL12 signaling inhibitor may be about 100 mg/kg per week. In one embodiment, the dose of the CXCL12 signaling inhibitor may be about 110 mg/kg per week. In one embodiment, the dose of the CXCL12 signaling inhibitor may be about 120 mg/kg per week. In one embodiment, the dose of the CXCL12 signaling inhibitor CXCL12 signaling inhibitor CXCL12 signaling inhibitor may be about 130 mg/kg per week. In one embodiment, the dose of the CXCL12 signaling inhibitor may be about 140 mg/kg per week. In one embodiment, the dose of the CXCL12 signaling inhibitor may be about 150 mg/kg per week. In one embodiment, the dose of the CXCL12 signaling inhibitor may be about 160 mg/kg per week. In one embodiment, the dose of the CXCL12 signaling inhibitor may be about 170 mg/kg per week. In one embodiment, the dose of the CXCL12 signaling inhibitor may be about 180 mg/kg per week. In one embodiment, the dose of the CXCL12 signaling inhibitor may be about 190 mg/kg per week. In one embodiment, the dose of the CXCL12 signaling inhibitor may be about 200 mg/kg per week. In one embodiment, the dose of the CXCL12 signaling inhibitor may be about 210 mg/kg per week. In one embodiment, the dose of the CXCL12 signaling inhibitor may be about 220 mg/kg per week. In one embodiment, the dose of the CXCL12 signaling inhibitor may be about 230 mg/kg per week. In one embodiment, the dose of the CXCL12 signaling inhibitor may be about 240 mg/kg per week. In one embodiment, the dose of the CXCL12 signaling inhibitor may be about 250 mg/kg per week. In one embodiment, the dose of the CXCL12 signaling inhibitor may be about 260 mg/kg per week. In one embodiment, the dose of the CXCL12 signaling inhibitor may be about 270 mg/kg per week. In one embodiment, the dose of the CXCL12 signaling inhibitor may be about 280 mg/kg per week. In one embodiment, the dose of the CXCL12 signaling inhibitor may be about 290 mg/kg per week. In one embodiment, the dose of the CXCL12 signaling inhibitor may be about 300 mg/kg per week. In one embodiment, the dose of the CXCL12 signaling inhibitor may be about 310 mg/kg per week. In one embodiment, the dose of the CXCL12 signaling inhibitor may be about 320 mg/kg per week. In one embodiment, the dose of the CXCL12 signaling inhibitor may be about 330 mg/kg per week. In one embodiment, the dose of the CXCL12 signaling inhibitor may be about 340 mg/kg per week. In one embodiment, the dose of the CXCL12 signaling inhibitor may be about 350 mg/kg per week.

In one aspect of the invention, the CXCL12 signaling inhibitor and the anti-cancer agent(s), e.g., chemotherapeutic agent(s), may be administered sequentially. That is, the CXCL12 signaling inhibitor may be administered for a period of time sufficient to have an anti-chemorepellent effect, and the anti-cancer agent, e.g., chemotherapeutic agent, may be subsequently administered.

In one aspect of the invention, administration of the CXCL12 signaling inhibitor may be pulsatile. In one embodiment, an amount of CXCL12 signaling inhibitor may be administered about every 1 hour to about every 24 hours, for example about every 1 hour, 2 hours, 3 hours, 4 hours, 5 hours, 6 hours, 7 hours, 8 hours, 9 hours, 10 hours, 11 hours, 12 hours, 13 hours, 14 hours 15 hours, 16 hours, 17 hours, 18 hours, 19 hours, 20 hours, 21 hours, 22 hours, 23 hours, or 24 hours, and/or any range and/or amount therein. In one embodiment, an amount of CXCL12 signaling inhibitor may be administered about every one day to about 14 days (e.g., about every 1 day, 2 days, 3 days, 4 days, 5 days, 6 days, 7 days, 8 days, days, 10 days, 11 day, 12, day, 13, days or 14 days and/or any range and/or amount therein).

In one aspect of the invention, doses of the CXCL12 signaling inhibitor may be administered in a pulsatile manner for a period of time sufficient to have an anti-chemorepellent effect (e.g. to attenuate the chemorepellant effect of the tumor cell). In one embodiment, the period of time sufficient to have an anti-chemorepellent effect may be between about 1 day and about 14 days. For example, the period of time sufficient to achieve an anti-chemorepellent effect may be about 1 day, 2 days, 3 days, 4 days, 5 days, 6 days, 7 days, 8 days, 9 days, 10 days, 11 day, 12, day, 13, days or 14 days.

In one aspect of the invention, the anti-cancer agent, e.g., chemotherapeutic agent, may be administered after the period of time of administration of CXCL12 signaling inhibitor. In one embodiment, the anti-cancer agent is administered during a period of time wherein the chemorepellent effect of the cancer cells/tumor is attenuated by the CXCL12 signaling inhibitor. The length of time and modes of administration of the anti-cancer agent will vary, depending on the anti-cancer agent used, type of tumor being treated, condition of the patient, and the like. Determination of such parameters is within the capability of the skilled clinician.

In one embodiment, administration of the CXCL12 signaling inhibitor and the anti-cancer agent, e.g., chemotherapeutic agent, may be alternated. In some embodiments, administration of the CXCL12 signaling inhibitor and the anti-cancer agent, e.g., chemotherapeutic agent, may be alternated until the condition of the patient improves. Improvement includes, without limitation, reduction in size of the tumor and/or metastases thereof, elimination of the tumor and/or metastases thereof, remission of the cancer, and/or attenuation of at least one symptom of the cancer.

In one aspect, this invention relates to a method for treating a tumor in a mammal, which tumor expresses CXCL12 at a concentration sufficient to produce a chemorepellent effect, the method comprising administering to said mammal an effective amount of a CXCL12 signaling inhibitor for a sufficient period of time so as to inhibit said chemorepellent effect, followed by administering to said mammal at least one anti-cancer agent, e.g., chemotherapeutic agent. In one embodiment, the cancer cell may be a solid tumor cell. In one embodiment, the cancer cell may be an ovarian cancer cell. In one embodiment, the cancer cell may be an epithelial ovarian cancer cell that has progressed to a recurrent platinum resistant disease. In one embodiment, the anti-cancer agent, e.g., chemotherapeutic agent, may be administered within about 3 days of completion of administration of the CXCL12 signaling inhibitor. In one embodiment, the anti-cancer agent, e.g., chemotherapeutic agent, may be administered within about 1 day of completion of administration of the CXCL12 signaling inhibitor. In some embodiments, the time between completion of administration of the CXCL12 signaling inhibitor and beginning the administration of the chemotherapeutic agent can be less than one day. In some embodiments, the chemotherapeutic agent may be administered within about 1 day up to about 14 days of completion of administration of the CXCL12 signaling inhibitor. In some embodiments, the time between completion of administration of the CXCL12 signaling inhibitor and beginning the administration of the chemotherapeutic agent can about one day to about four weeks. Thus, in some embodiments, the time between completion of administration of the CXCL12 signaling inhibitor and beginning the administration of the chemotherapeutic agent may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or any range or value therein.

A variety of administration routes are useful with this invention. The methods of the invention, generally speaking may be practiced using any mode of administration that is medically acceptable, meaning any mode that produces effective levels of the active compounds without causing clinically unacceptable adverse effects.

In one embodiment, the CXCL12 signaling inhibitors and/or chemotherapeutic agents may be administered directly to a subject. Generally, the inhibitors and/or agents will be suspended in a pharmaceutically-acceptable carrier (e.g., physiological saline) and administered orally or by intravenous infusion, or administered topically, subcutaneously, intramuscularly, intrathecally, intraperitoneally, intrarectally, intravaginally, intranasally, intragastrically, intradermally, intratracheally, intrapulmonarily or by infusion. In another embodiment, the intratracheal or intrapulmonary delivery can be accomplished using a standard nebulizer, jet nebulizer, wire mesh nebulizer, dry powder inhaler, or metered dose inhaler. They can be delivered directly to the site of the disease or disorder, such as ovaries, lungs, kidney, or intestines or directly into a tumor.

In one embodiment, the CXCL12 signaling inhibitors and/or chemotherapeutic agents may be administered proximal to (e.g., near or within the same body cavity as) the tumor, e.g., into the peritoneal or pleural cavity, or topically, e.g., to the skin or a mucosal surface, e.g., to the ovaries. In one embodiment, the inhibitors and/or agents may be administered directly into the tumor or into a blood vessel feeding the tumor. In one embodiment, the inhibitors and/or agents are administered systemically. In a further embodiment, the inhibitors and/or agents may be administered by microcatheter, or an implanted device, or an implanted dosage form.

The term “parenteral” includes subcutaneous, intravenous, intramuscular, or infusion. Oral administration may be used for prophylactic treatment for the convenience to the patient as well as the dosing schedule. When peptides are used therapeutically, in certain embodiments a route of administration may be by pulmonary aerosol. Techniques for preparing aerosol delivery systems containing peptides are well known to those of skill in the art. Generally, such systems utilize components that will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing antibody or peptide aerosols without resort to undue experimentation.

Compositions suitable for oral administration may be presented as discrete units, such as capsules, tablets, lozenges, each containing a predetermined amount of the active agent(s) (e.g., the chemotherapeutic agent and/or anti-chemorepellent agents (e.g., CXCL12 signaling inhibitors)). Other compositions include suspensions in aqueous liquids or non-aqueous liquids such as a syrup, elixir or an emulsion.

Preparations for parenteral administration may include sterile aqueous or non-aqueous solutions, suspensions, and emulsions. Examples of non-aqueous solvents include, but are not limited to, propylene glycol, polyethylene glycol, vegetable oils such as olive oil, and injectable organic esters such as ethyl oleate. Aqueous carriers include, but are not limited to, water, alcoholic/aqueous solutions, emulsions or suspensions, including saline and buffered media. Parenteral vehicles include, for example, sodium chloride solution, Ringer's dextrose, dextrose and sodium chloride, lactated Ringer's and/or fixed 25 oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Lower doses may result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day may be used to achieve appropriate systemic levels of compounds.

In one embodiment, a CXCL12 signaling inhibitor may be administered parenterally. In one embodiment, a CXCL12 signaling inhibitor may be administered via microcatheter into a blood vessel proximal to a tumor. In one embodiment, a CXCL12 signaling inhibitor may be administered via microcatheter into a blood vessel within a tumor. In one embodiment, a CXCL12 signaling inhibitor may be administered subcutaneously. In one embodiment, a CXCL12 signaling inhibitor may be administered intradermally.

Other delivery systems can include, for example, time-release, delayed release or sustained release delivery systems. Such systems can avoid repeated administrations of the CXCL12 signaling inhibitor, increasing convenience to the subject and the physician. Many types of release delivery systems are available and known to those of ordinary skill in the art. They include polymer base systems including, but not limited to, poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like.

In one embodiment, a CXCL12 signaling inhibitormay be administered in, a time-release, delayed release or sustained release delivery system. In one embodiment, the time-release, delayed release or sustained release delivery system comprising the CXCL12 signaling inhibitormay be inserted directly into the tumor. In one embodiment, the time-release, delayed release or sustained release delivery system comprising the CXCL12 signaling inhibitormay be implanted in the patient proximal to the tumor. Additional implantable formulations are described, for example, in U.S. Patent App. Pub. No. 20080300165, which is incorporated herein by reference in its entirety.

In addition, some embodiments of the invention include pump-based hardware delivery systems, some of which are adapted for implantation. Such implantable pumps include controlled-release microchips. In some embodiments, a controlled-release microchip useful with the invention may be that described in Santini, J T Jr. et al., Nature, 1999, 397:335-338, the contents of which are expressly incorporated herein by reference.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

### Methods of Treatment

In one aspect of this invention is provided a method for treating cancer in a subject in need thereof by administration of an inhibitor of CXCL12 signaling in combination with a chemotherapeutic agent. In some embodiments, the CXCL12 signaling inhibitor may be administered in combination with a subtherapeutic amount of a chemotherapeutic agent.

In an aspect of this invention, a CXCL12 signaling inhibitor, e.g., AMD3100, in combination with a subtherapeutic amount of a chemotherapeutic agent, e.g., paclitaxel, which includes TAXOL® and ABRAXANE®, may be administered to a subject having a tumor that expresses a chemokine in sufficient amounts to produce a chemorepellent effect. The subject may be, e.g., a subject having an ovarian cancer that expresses a chemokine in sufficient amounts to produce a chemorepellent effect and/or has progressed to recurrent platinum resistant disease. In an aspect of the invention, an inhibitor of CXCL12 signaling may be administered to a subject in need thereof in an amount that enhances the penetration of immune cells into a tumor and the chemotherapeutic agent is administered in a subtherapeutic amount at the same time, before or after administration of the CXCL12 signaling inhibitor.

An embodiment of this invention includes a method for killing a cancer cell expressing an amount of a chemokine sufficient to produce a chemorepellent effect in a subject in need thereof, which method comprises:

a) contacting the cancer cell with an amount of a CXCL12 signaling inhibitor for a sufficient period of time to inhibit the chemorepellent effect and to increase migration of immune cells to the cancer cell;
b) contacting the cancer cell with a subtherapeutic amount of a chemotherapeutic agent, and
c) optionally repeating steps a) and b) as necessary to kill said cell.

In an embodiment of the methods of this invention the CXCL12 signaling inhibitor and chemotherapeutic agent are administered at the same time/concurrently or sequentially. In an embodiment of the methods of this invention the CXCL12 signaling inhibitor may be administered before administering the chemotherapeutic agent. In an embodiment of the methods of this invention the CXCL12 signaling inhibitor may be administered from about 1 day to about 14 days (i.e., administration may be completed up to 14 days) before administering the chemotherapeutic agent.

In some embodiments, contacting of the cancer cell with the CXCL12 signaling inhibitor may be periodic.

In some embodiments, when steps (a) and (b) are repeated, they may be repeated, for example, at least one time. In some embodiment, steps (a) and (b) may be repeated more than one time (e.g., about 2 to about 10 times or more, e.g., about 1, 2, 3, 4, 5, 6, 7, 8, 9, or 10 times, or more).

In an embodiment, CXCL12 signaling inhibitors and anti-cancer agent(s), e.g., chemotherapeutic agents, e.g., a taxane, a paclitaxel including, but not limited to, TAXOL® and/or ABRAXANE®, may be administered sequentially. For example, a CXCL12 signaling inhibitor may be administered for a period of time sufficient to reduce or attenuate the chemorepellent effect of the tumor (e.g., about every 1 hour to about every 24 hours for 1 day to about 14 days up to about 4 weeks), e.g. such that the CXCL12 signaling inhibitor has an anti-chemorepellent effect (e.g., increase penetration and/or migration of an immune cell into and/or to a tumor); the anti-cancer agent, e.g., chemotherapeutic agent, may then be administered for a period of time during which the chemorepellent effect of the tumor is reduced or attenuated. In one embodiment, the CXCL12 signaling inhibitor and chemotherapeutic agent may be administered sequentially in an alternating manner at least until the condition of the subject improves. Improvement of the condition of the subject includes, without limitation, reduction in tumor size, a reduction in at least one symptom of the cancer, elimination of the tumor and/or metastases thereof, increased survival of the subject, and the like.

In one embodiment, a CXCL12 signaling inhibitor and/or a chemotherapeutic agent may be administered intravenously, subcutaneously, orally, or intraperitoneally. In some embodiments, a CXCL12 signaling inhibitor may be administered proximal to (e.g., near or within the same body cavity as) the tumor. In one embodiment, the CXCL12 signaling inhibitor may be administered directly into the tumor or into a blood vessel feeding the tumor. In one embodiment, a CXCL12 signaling inhibitor may be administered systemically. In a further embodiment, a CXCL12 signaling inhibitor may be administered by microcatheter, an implanted device, and/or an implanted dosage form.

A CXCL12 signaling inhibitor may be any such inhibitor known in the art. In one embodiment, a CXCL12 signaling inhibitor is a CXCL12 signaling inhibitor as described in U.S. Patent Application Publication No. 2008/0300165, which is hereby incorporated by reference in its entirety. In some embodiments, the CXCL12 signaling inhibitor may be AMD3100 (mozobil/plerixafor), AMD11070, AMD12118, AMD11814, AMD13073, FAMD3465, C131, BKT140, CTCE-9908, KRH-1636, KRH-2731, KRH-3955, TC14012, BMS-936564/MDX-1338, LY2510924, GSK812397, T-20, T-22, T-140, TE-14011, T-14012, TN14003, TAK-779, AK602, SCH-351125, Tannic acid, NSC 651016, thalidomide, GF 109230X, and/or an antibody that interferes with ability of a chemorepellent to act in a chemorepellent manner. In one embodiment, the CXCL12 signaling inhibitor may be an antibody that interferes with binding of the chemokine to its receptor. In representative embodiments, a CXCL12 signaling inhibitor useful with this invention may be AMD3100.

A chemokine that is expressed by the cancer cells in an amount sufficient to produce a chemorepellent effect may include, but is not limited to, CXCL12 or interleukin 8. In one embodiment, the chemokine is secreted by the cell or tumor, such that the chemorepellent effect is present in the tumor microenvironment. In one embodiment, the concentration of the chemokine in the tumor microenvironment may be at least about 100 nM (e.g., at least about 100, 125, 150, 175, 200, 225, 250, 275, 300, 325, 350, 375, 400, 425, 450, 475, 500 nM, or more, and any range or value therein) prior to treatment with the CXCL12 signaling inhibitor. In one embodiment, the chemokine is CXCL12 or IL-8. In a some embodiments, the chemokine is CXCL12.

In an embodiment of this invention the cancer cell may be a solid tumor cell, an ovarian cancer cell, e.g., an epithelial ovarian cancer cell, a fallopian tube cancer cell, or a primary peritoneal cancer cell. The ovarian cancer cell may be, e.g., a cancer cell that has progressed to platinum resistance.

Also an embodiment of this invention is a method for killing a cancer cell in a solid tumor expressing a chemokine at a concentration sufficient to produce a chemorepellent effect in a subject, which method comprises, a) administering an effective amount of a CXCL12 signaling inhibitor into the tumor for a sufficient time to increase penetration of an immune cell into the tumor; and d) subsequently administering a subtherapeutic amount of the chemotherapeutic agent to the subject, thereby killing the cancer cell.

An additional embodiment of this invention is a method for treating a tumor expressing a chemokine at a concentration sufficient to produce a chemorepellent effect in a subject, which method comprises: a) injecting or infusing an amount of a CXCL12 signaling inhibitor into said tumor for a sufficient time to increase penetration of immune cells into the tumor; and b) subsequently administering subtherapeutic amount of the chemotherapeutic agent to the patient, wherein the subtherapeutic amount of the chemotherapeutic provides a therapeutic effect on the tumor that is enhanced as compared to a subtherapeutic amount administered without the CXCL12 signaling inhibitor, thereby treating the subject.

Another embodiment of this invention is a method for enhancing the therapeutic effect of a chemotherapeutic agent on a tumor expressing an amount of a chemokine sufficient to produce a chemorepellent effect, which method comprises, a) administering an effective amount of a CXCL12 signaling inhibitor to a subject having the tumor for a sufficient time to increase penetration of immune cells into the tumor; and b) administering subtherapeutic amount of the chemotherapeutic agent to the subject.

Another embodiment of this invention is a method for enhancing the therapeutic effect of a chemotherapeutic agent on a tumor expressing an amount of a chemokine sufficient to produce a chemorepellent effect, which method comprises, a) selecting a subject having a tumor expressing an amount of a chemokine sufficient to produce a chemorepellent effect, b) administering an effective amount of a CXCL12 signaling inhibitor to a subject having the tumor for a sufficient time to increase penetration of immune cells into the tumor; and c) administering a subtherapeutic amount of the chemotherapeutic agent to the subject, wherein the therapeutic effectiveness of the subtherapeutic amount of the chemotherapeutic agent is enhanced as compared to a control (e.g., the subtherapeutic amount administered without the CXCL12 signaling inhibitor).

An embodiment of this invention is a method for increasing immune cell migration into a tumor which method comprises

a) identifying a tumor having a chemorepellent property whereby immune cells are repelled from the tumor,
b) contacting the tumor with an amount of a CXCL12 signaling inhibitor for a sufficient period of time to inhibit the chemorepellent effect;
c) contacting the tumor with a subtherapeutic amount of a chemotherapeutic agent, and
d) optionally repeating steps b) and c),
wherein the migration of immune cells into the tumor is increased.

In some embodiments, the tumor may be periodically contacted with the CXCL12 signaling inhibitor as described herein.

In some embodiments, when steps (b) and (c) are repeated, they may be repeated, for example, at least one time. In some embodiment, steps (a) and (b) may be repeated more than one time (e.g., about 1, 2, 3, 4, 5, 6, 7, 8, 9 times or more).

In embodiments of this invention, the CXCL12 signaling inhibitor and chemotherapeutic agent may be administered to a subject in need thereof having a tumor which expresses a chemokine in an amount sufficient to produce a chemorepellent effect. In embodiments of this invention, such tumor may be a solid tumor. In embodiments of this invention, such tumor may be an ovarian tumor. The subject may be a subject who has or has had a recurrence of epithelial ovarian cancer, primary peritoneal cancer, or fallopian tube cancer.

An embodiment of this invention is a method for killing a cancer cell in a solid tumor expressing a chemokine at a concentration sufficient to produce a chemorepellent effect in a subject in need thereof, which method comprises a) administering an effective amount of a CXCL12 signaling inhibitor into said tumor for a sufficient time to increase penetration of an immune cell into the tumor; and b) subsequently administering a subtherapeutic amount of the chemotherapeutic agent to the subject.

An embodiment of this invention is a method for treating a tumor expressing a chemokine at a concentration sufficient to produce a chemorepellent effect in a subject in need thereof, which method comprises, a) injecting or infusing an amount of a CXCL12 signaling inhibitor into said tumor for a sufficient time to increase penetration of an immune cell into the tumor; and b) subsequently administering subtherapeutic the chemotherapeutic agent to the subject.

An embodiment of this invention is a method for enhancing the therapeutic effect of a chemotherapeutic agent on a tumor expressing an amount of a chemokine sufficient to produce a chemorepellent effect, which method comprises, a) administering an effective amount of a CXCL12 signaling inhibitor to a subject having the tumor for a sufficient time to increase penetration of an immune cell into the tumor; and b) administering subtherapeutic amount of the chemotherapeutic agent to the subject.

An embodiment of this invention is a method for enhancing the therapeutic effect of a chemotherapeutic agent on a tumor expressing an amount of a chemokine sufficient to produce a chemorepellent effect, which method comprises, a) selecting a subject having a tumor expressing an amount of a chemokine sufficient to produce a chemorepellent effect, b) administering an effective amount of a CXCL12 signaling inhibitor to the subject or a sufficient time to increase penetration of immune cells into the tumor; and c) administering a subtherapeutic amount of the chemotherapeutic agent to the subject.

In some embodiments of the invention, the CXCL12 signaling inhibitor may be AMD3100.

In some embodiments of the invention, the chemotherapeutic agent may be a taxane.

In some embodiments of the invention, the chemotherapeutic agent may be paclitaxel.

In some embodiments of the invention, the chemotherapeutic agent may be TAXOL®.

In some embodiments of the invention, the chemotherapeutic agent may be ABRAXANE®.

In some embodiments of the invention, the CXCL12 signaling inhibitor may be AMD3100 and the chemotherapeutic agent may be a paclitaxel.

Having described the present invention, the same will be explained in greater detail in the following examples, which are included herein for illustration purposes only, and which are not intended to be limiting to the invention. There are a variety of alternative techniques and procedures available to those of skill in the art which would similarly permit one to successfully perform the intended invention.

### EXAMPLES

### Example 1

Patients with recurrent, ovarian, fallopian tube or primary peritoneal cancer are treated with AMD3100 in combination with Taxol using a Q 28 day schedule, to: identify the maximum tolerated dose of AMD3100 in combination with taxol in patients with recurrent, ovarian, fallopian tube or primary peritoneal cancer,

assess the response rate of Taxol-AMD3100 combination,
assess progression-free survival and overall survival in patients with recurrent ovarian, fallopian tube, or peritoneal cancer, treated with Taxol and AMD3100,
assess toxicities of treatment with combination Taxol and AMD3100 in recurrent ovarian, fallopian tube and peritoneal cancer, and
characterize the pharmacokinetics (PK) of Taxol and AMD3100 when used in combination.

Subjects are recruited by addressing the major inclusion and exclusion criteria:

Inclusion Criteria:
Patients are diagnosed with a recurrence of epithelial ovarian cancer, primary peritoneal cancer, or fallopian tube cancer.
The following histologic subtypes are eligible: papillary serous, endometrioid, mucinous, clear cell, adenocarcinomas, transitional, carcinosarcoma, and mixtures of the above. Patients with sarcomatous, stromal, or germ cell elements in their cancers are not eligible. Patients must have at least one measurable lesion according to RECIST criteria via CT or MRI scan. CT of the chest should be performed if any known disease is present in the chest (i.e. pleural effusions, lung metastases, pleural-based metastases). Pleural effusions, ascites, bone metastases, CA125 tumor markers, and lesions located in previously radiated areas are not considered measurable. Eastern Cooperative Group (ECOG) Performance status of 0, 1, 2.
Age ≥18 years of age
Life expectancy ≥12 weeks
Baseline laboratory values must meet all of the following:
ANC>1500/mm³
Platelets >100,000/mm³
Total bilirubin <1.5 ULN (upper limit of normal)
Calculated creatinine clearance >45 ml/min (using Cockcroft-Gault
formula as described in Section 6.4.4)
Creatinine of <1.5×ULN
ALT/AST<3×ULN (no liver mets)
ALT/AST<5×ULN (with liver mets)
Complete recovery from completion of previous chemotherapy or biologic therapy Women of childbearing potential must have a negative pregnancy test within 7 days prior to initiating chemotherapy on trial and must agree to practice an effective method of birth control, such as an intrauterine device, tubal ligation, or oral contraceptives, during the study and for six months after their last treatment.

Exclusion Criteria:
Prior pelvic radiotherapy >25% of bone marrow
Any uncontrolled medical problem that in the opinion of the investigator would preclude safe administration of the study drugs.
Past history of bone marrow transplantation or stem cell support.
Patient with known history of CNS metastasis is ineligible unless the patient has had treatment with surgery or radiation therapy, is neurologically stable, and does not require oral or intravenous corticosteroids or anticonvulsants.
A history of prior malignancy except for adequately treated carcinoma in situ of the uterine cervix, incidental stage I endometrial cancer, basal cell or squamous cell skin cancer, or breast cancer (invasive or ductal carcinoma in situ) for which the patient has been disease-free for at least three years.
Routine prophylactic use of G-CSF or GM-CSF within two weeks prior to study entry.
Clinically significant cardiac disease, as defined by:
History of unstable angina within six months of study entry
History of symptomatic ventricular arrhythmias
History of congestive heart failure
History of myocardial infarction within six months of study entry
Uncontrolled hypercalcemia or diabetes mellitus
Any signs of intestinal obstruction that interfere with bowel function and/or nutrition
Grade ≥2 peripheral neuropathy.
Participation in an investigational study within three weeks prior to study entry.
History of psychiatric disability or other central nervous system disorder as judged by the principal investigator that would be considered significant and that would preclude infotnied consent, safe administration of study medications, and affecting ability to comply with study procedures

### Study Design

A Phase I trial is used to identify and test the appropriate Phase II dose of Taxol-AMD3100 combination in patients with recurrent ovarian, fallopian tube and primary peritoneal cancer.

Phase I portion is conducted using a standard step-up/down dose escalation design. The MTD is defined as the highest dose with no more than one dose-limiting toxicity (DLT) occurring in any of the 6 patients treated at that dose level. The DLT is defined as any toxicity that is ≥grade 3, or failure to return to treatment criteria within 14 days. A cohort of 3 patients will be initially enrolled to the given dose level, and dose escalation will proceed as follows:

a) If no DLT toxicities are observed, then the dose is then escalated to the next dose level in the next group of 3 patients.
b) If 1 DLT criteria-meeting toxicity is observed, then up to 3 additional patients is then treated at the same dose level.
a. If no additional DLT are observed for the additional 3 patients, then the dose will be escalated in the next group of 3 patients.
b. If a second DLT is observed at the given dose level, treatment of patients at that dose level will cease, and treatment of up to 3 additional patients at the next lower dose level will begin.
c) If 2 or more DLT are observed in the initial 3 patients at any given dose level, then up to 3 additional patients are then treated at the next lower dose level.
a. If a second DLT is seen at the next lower dose level, then treatment of patients at that dose level will cease, and treatment of up to 3 additional patients on the next lower dose level will begin.
b. If no more than 1 DLT is seen after the next lower dose level has enrolled a total of 6 patients, then that dose level will be declared the MID.
d) This step-down process continues until a dose level is reached for which no more than one DLT on any of 6 patients treated at that dose level is observed; that level is then declared the MTD.
e) If more than 1 DLT is observed at the starting dose level (Dose Level 1), then dose levels is then decreased to Dose Level −1.
Using this model, the probability to escalate the dose if the true toxicity is >65% is less than 0.053, and the probability to escalate the dose if the true toxicity rate is 10% or less is greater than 0.90.

Primary endpoint is complete response (CR) or partial response (PR), and the secondary endpoints are progression free survival (PFS) and overall survival (OS) times.

All data analysis is based on intention to treat population. Safety analysis includes descriptive tabulation of the grade 2, 3, and 4 hematologic toxicities such as neutropenia, thrombocytopenia and anemia. Response rate is summarized with a 95% confidence interval based on Binomial probability model. In addition, the progression-free survival and overall survival is measured in each patient. Kaplan-Meier analysis is performed to determine the median times to progression and overall survival.

The primary objective of the pharmacokinetic studies is to determine whether or not the disposition of taxol and AMD3100 are affected by their concurrent administration.

### Example 2

Patients with recurrent, ovarian, fallopian tube or primary peritoneal cancer are treated with AMD3100 in combination with Taxol using a Q 28 day schedule as set forth in Example 1, however the patients in this example are treated with subtherapeutic amounts of paclitaxel or ABRAXANE® in combination with AMD 3100.

It is contemplated that the co-administration of paclitaxel, including TAXOL® and ABRAXANE® will result in an increase in the migration of immune cells into the tumor and/or improve the effectiveness of subtherapeutic amounts of the chemotherapeutic agents in the treatment of tumors that express a chemokine in amounts sufficient to produce a chemorepellent effect. It is contemplated that the co-administration of paclitaxel, including TAXOL® and ABRAXANE® will result in increased degradation of the tumor.

### Example 3

### Materials and Methods

Cell lines: ID8 cells, a clone of the MOSEC ovarian carcinoma TAXOL of C57BL/6 origin, and TOV-112D cells (ATCC, CRL11731) were cultured in DMEM supplemented with 10% FBS, 1% L-glutamine, and 1% Penicillin/Streptomycin at 37° C. and 5% CO₂.

Reagents: CyQuant Cell Proliferation kit (Invitrogen, C7026), and Paclitaxel (T7402) were purchased from Sigma Aldrich. AMD3100 (Plerixafor octahydrochioride) from Medchemexpress (HY-50912) or Abcam (ab1207180). Colony formation assays performed with SeaPlaque Agarose (Lonza, cat 50070).

Drug Titrations: TAXOL was titered to determine the concentrations used in combination with AMD3100. Cells were cultured in 96-well black assay plates with optically clear bottoms (500 and 2500 cells per well were tested), including a standard curve plate. ID8 cells were then exposed to increasing concentrations of paclitaxel (1, 0.5, 0.15, 0.05, 0.015, and 0.005 μM), and TOV cells were exposed to 15, 5, and 1 nM Taxol for 48 or 96 hr. Proliferation was measured by measuring the amount of dsDNA using CyQuant reagent and read on a Perkin Elmer Envision 2103. AMD3100 (50, 25, 10, 5, 2.5, and 1 μM) was titered in the same manner. Each condition was done in triplicate. Drugs were not replenished during any of the experiments. Data was analyzed in Prism 7. The concentrations that inhibited cell proliferation by 50% or the highest concentration with minimum to no effect on cell proliferation was used from each drug to treat tumor cells.

Simultaneous Delivery of Small Molecules and Chemotherapy: To determine the effect of AMD3100 alone or in combination with TAXOL, cell proliferation was measured by seeding 2500 TOV cells and 500 ID8 cell per well in a clear bottom 96-well black assay plates. AMD3100 (final concentrations: 10 μM for ID8 and 2.5 μM for TOV) and Paclitaxel (final concentrations: 15 and 5 nM for ID8 cells and 5 and 1 nM for TOV cells) were mixed with DMEM. 100 μL of the mixture was added to the cells. Each condition was done in triplicate. Plates were incubated at 37° C. with 5% CO2 for 72 hr (ID8) and 96 hr (TOV-112D). Standard curve plates were washed the same day the experiment was done. Plates were stored at −80° C. after washing and proliferation rate was measured then by CyQuant assay.

Sequential Delivery of Small Molecules and Chemotherapy: ID8 cells were exposed to AMD3100 in a 6 well plate (2.5×10⁵ cells/well) for 72 hr. Cells were then washed and counted. Cell viability was checked by propidium iodide. For each condition, 500 cells per well of viable ID8 cells were then seeded into a 96 well black assay plate. ID8 cells were then incubated in DMEM containing a carrier, AMD3100 10 μM, TAXOL 0.005 μM, or both for another 72 hr. For TOV-112D cells, initial 72 hr incubation was with AMD3100 5 μM, followed by 96 hr incubation in media containing AMD3100 5 μM, TAXOL 1 nM, or both. At the conclusion of the experiment, plates were washed and dsDNA was measured via CyQuant assay.

ID8 and TOV Colony Formation in The Presence of Small Molecules and/or Chemotherapy: Bottom Layer Hard Agar: 2×DMEM and 0.8% agar stock was prepared and adjusted to 42° C. in a water bath for 30 min. The agar and 2×DMEM were mixed (1:2). 500 μL of the mixture was then added to each well of a 24 well plate, and the plate was rocked to distribute evenly and allowed to solidify at room temperature in a tissue culture hood. Upper Layer Soft Agar: 0.6% agar stock was prepared adjusted to 42° C. in a water bath for 30 min. During this time, ID8 cells were trypsinized, and 40,000 cells per mL suspension in 1×DMEM was made. Cell suspensions were then mixed with soft agar stock (1:2) and 500 μL of the mixture was then added to each well. Each condition was in triplicate.

Drug-DMEM stocks were made to achieve the proper concentrations in 1.1 mL, the total volume of each well: AMD3100 (10 μM and 1 μM for ID8 cells, 5 μM and 2.5 μM for TOV cells) and TAXOL (0.015 μM and 0.005 μM). 100 μL of each drug was added to the relative wells in triplicate. Plates were then incubated in a humidified incubator with 50% CO₂ for 20-30 days. At the end of the experiment, each well was stained with 0.005% crystal violet in 15% ethanol (140 μL per well) and plates were incubated again for several hours then photos were taken using a microscope. ID8 Colonies equal or bigger 2 mm and TOV colonies equal or bigger than 5 mm were counted using Image J software.

CXCR4, CXCR7, and CXCL12 expression in ovarian cancer cells: ID8 or TOV-112D cells were plated at 750,000 cells/well in 6-well plates. Conditions were WT, WT+AMD3100 (10 μM for ID8/5 μM for TOV-112D, MedChemExpress HY-50912 and AbCam ab1207180) 16 hrs, and WT+AMD3100 4 hrs. 2× drug stocks were made in complete DMEM, final volume per well was 2 mL. GolgiPlug (BD Pharmingen) was added to all wells 3 hrs prior to harvesting according to manufacturer's protocol. CXCL12, CXCR4, and CXCR7 expression was measured by Flow Cytometry. Antibodies: anti-h/mCXCL12/SDF-1 APC (IC350A, RandD Systems), anti-mCXCR4/CD184 PE (551966, BD Bioscience), anti-hCXCR4/CD184 PE (306506, BioLegend), anti-h/mCXCR7 PE-Cy7 (331115, BioLegend).

Flow Cytometry: Cells were harvested using enzyme free dissociation buffer (Thermo Fisher Scientific 13151014) and counted using Trypan Blue viability dye (Corning). Equal numbers of cells were labeled with antibody for surface markers. Thereafter cells were stained for viability and where needed with secondary antibody. Fixation/Permeabilization to stain for intracellular markers was achieved with the FoxP3 Fix/Perm Kit (eBioscience) after which cells were stained with intracellular antibody. All incubation steps were performed at 4° C. on a rocker in the dark. Cells were washed 2× before analysis in cell staining buffer consisting of PBS containing 0.5% BSA, 0.02% Sodium Azide, and 2 mM EDTA. Analysis of samples in duplicate was done with a BD Fortessa collecting a minimum of 10,000 events per sample and using FlowJo software.

### Example 4

### CXCR4, CXCR7, and CXCL12 Expression by IDS and TOV-112D Ovarian Cancer Cells

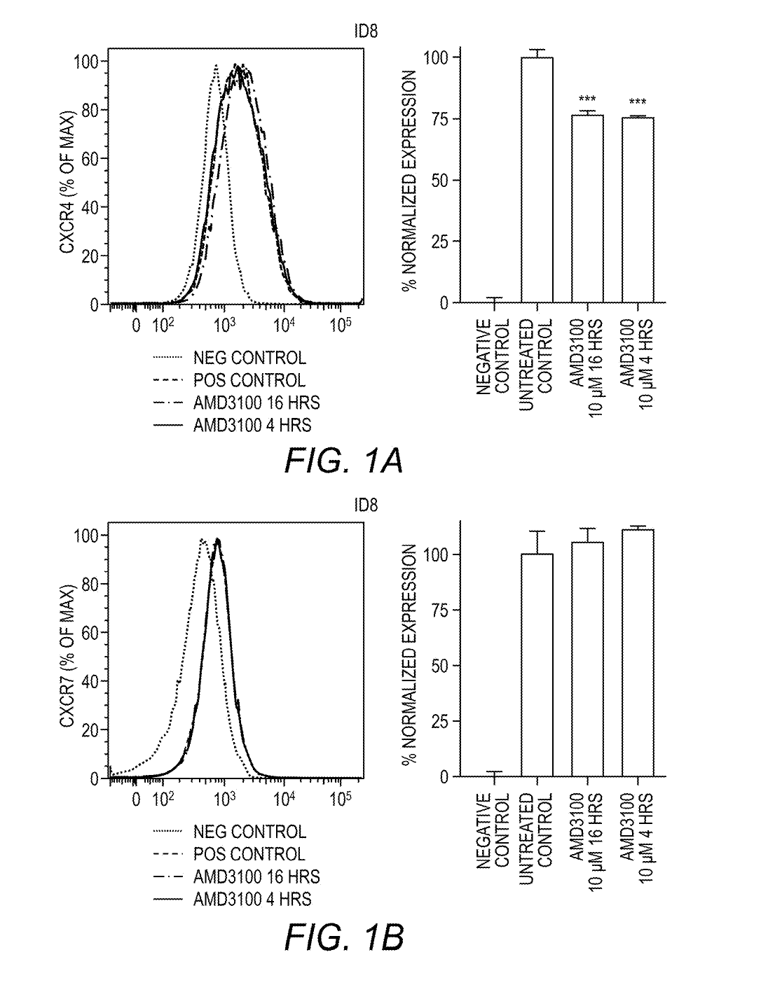

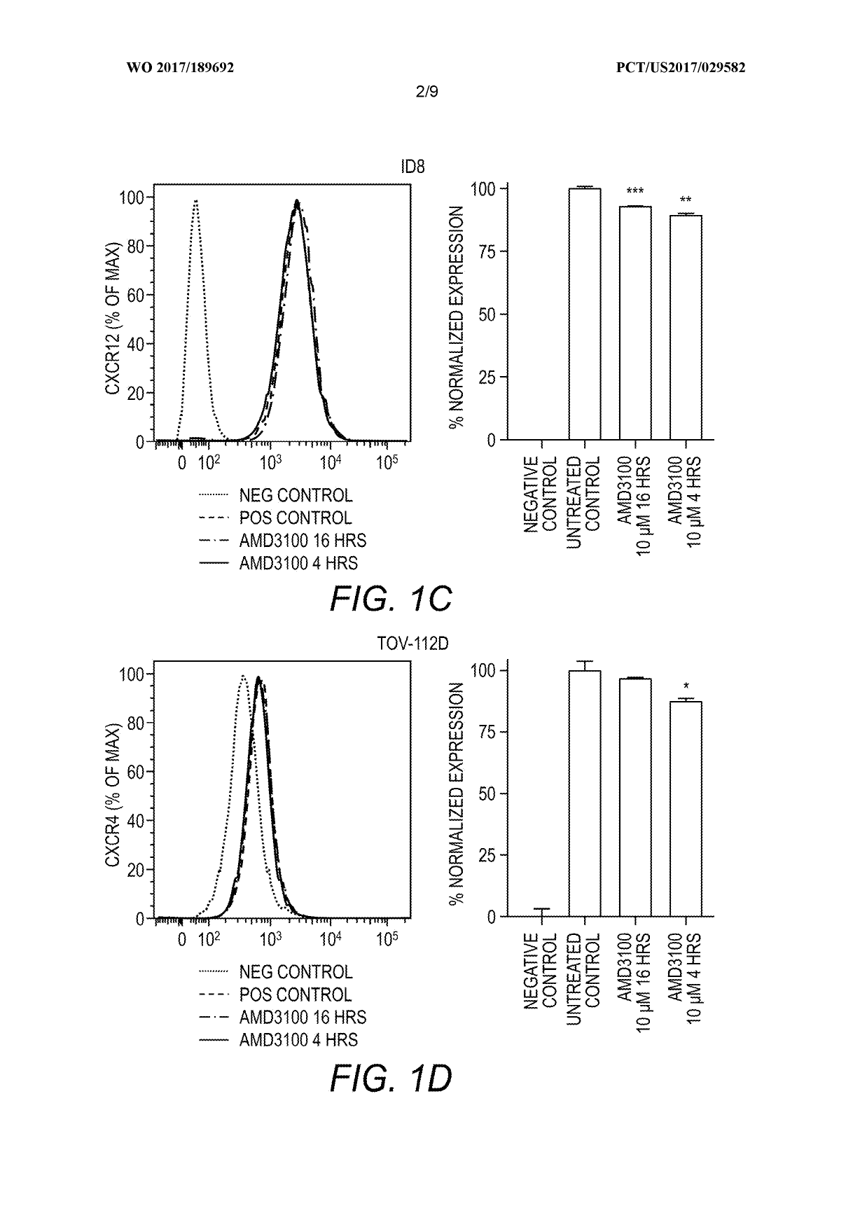

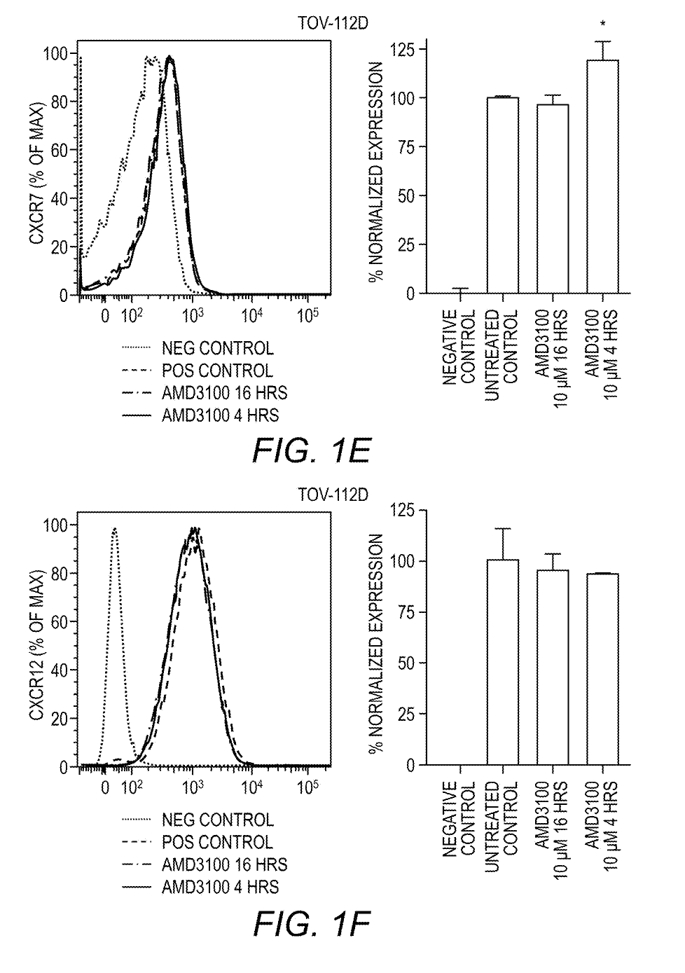

The expression of CXCR4, CXCR7, and CXCL12 was determined by flow cytometry for both ID8 and TOV-112D cells. Both ID8 and TOV-12D cells were positive for labeling with antibodies specific to CXCR4, modestly positive for CXCR7 and expressed a high amount of intracellular CXCL12 (FIGS. 1A-IF). Treatment of ID8 and TOV-112D cells with the CXCR4 antagonist AMD3100 for either 4 or 18 hours did not significantly alter the surface expression of CXCR4 and CXCR7, nor overall expression of CXCL12 (FIGS. 1A-IF). Normalization of the data, setting the negative control as 0 and untreated cells at 100, revealed some changes for the various AMD3100 treatment conditions. ID8 cells incubated with AMD3100 for either 4 or 18 hours were reduced in expression of CXCR4 by 24.5% and 23.5% respectively, CXCR7 expression increased by 6%-11%, and CXCL12 expression was reduced by 9-10% (FIGS. 1A-1F). TOV-112D cells treated in a similar manner were reduced for CXCR4 by 13% and 4% after 4 or 18 hours respectively, CXCR7 expression increased by 20% after 4 hours and then returned to basal levels, and CXCL12 by 5-7% (FIG. 1B). Expression of CXCL12 in both ID8 and TOV-112D cells was modestly reduced by AMD3100 treatment, regardless of incubation period. Conversely, acute AMD3100 treatment reduces expression of CXCR4 in both ID8 and TOV-112D cells, while prolonged incubation preferentially reduces expression in ID8 cells.

### Example 5

### AMD3100 in Combination with Low-Dose TAXOL Limits ID8 and TOV-112D Cell Proliferation

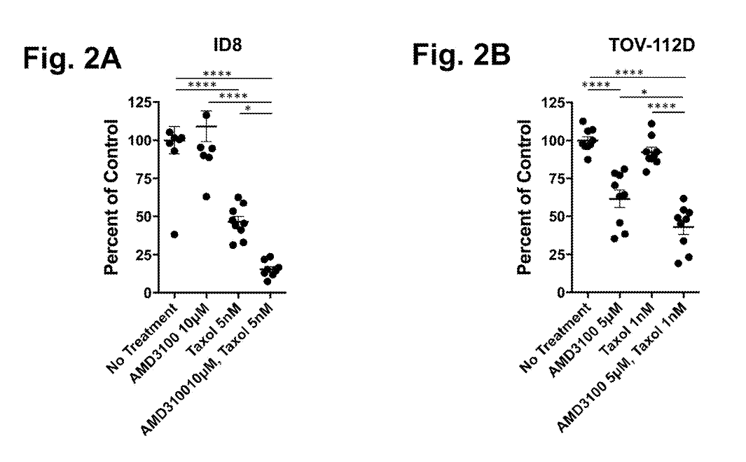

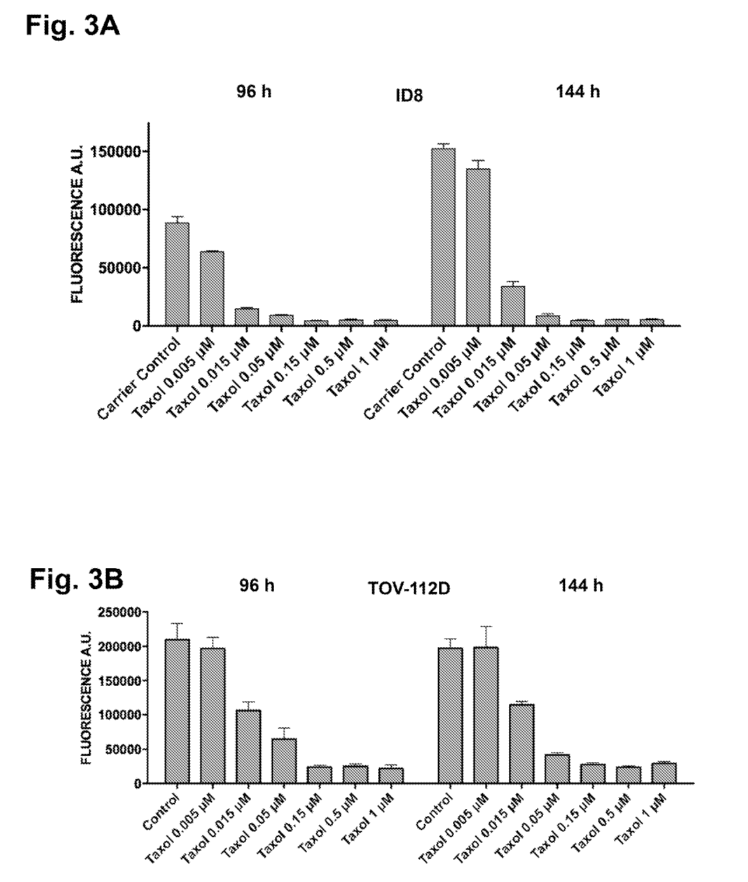

The impact of AMD3100 on 1D8 murine ovarian cancer cell proliferation in vitro was evaluated and after 72 hours it was observed that AMD3100 (10 μM) treatment alone did not significantly inhibit ID8 cell growth (FIG. 2A). We next determined the TAXOL concentration that are sufficient to inhibit 1D8 cell proliferation by 50% after 72 hrs (IC₅₀-0.005 μM, (FIG. 2A, FIG. 3A-3B). Co-incubation with both AMD3100 (10 μM) and TAXOL (0.005 μM) significantly reduced ID8 cell proliferation by 80.7% (P value <0.0001) (FIG. 2A). Treatment of TOV-11D human ovarian cancer cells with 2.5 μM AMD3100 limited cell growth by 24.5%. We were not able to identify a dose of TAXOL that reproducibly inhibited TOV-112D proliferation by 50%, and therefore, 1 nM TAXOL was utilized, a concentration that did not significantly inhibit cell proliferation after 72 hours (FIG. 2B, FIG. 3A-3B). In combination, AMD3100 (2.5 μM) and TAXOL (1 nM) significantly reduced TOV-112D cell growth by 49.2% (P Value <0.0001) (FIG. 2B). Thus AMD3100 sensitizes both murine ID8 cells and human TOV-112D cells to simultaneous incubation with reduced doses of TAXOL.

### Example 6

### Sequential Delivery of AMD3100 Limits ID8 and TOV-112D Cell Proliferation

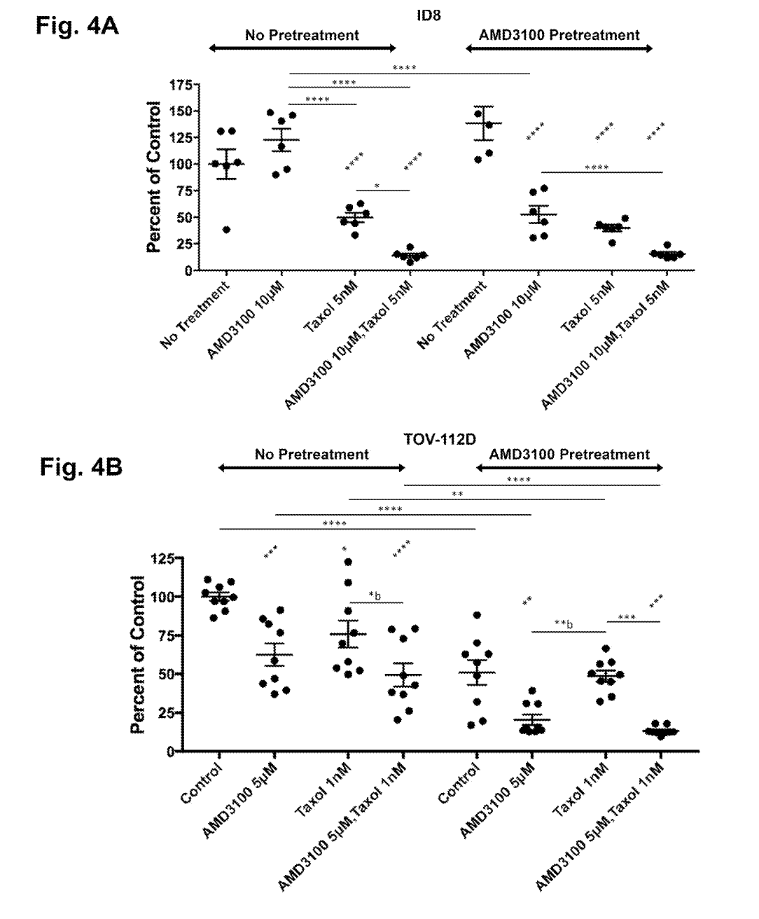

Recent reports indicate that for some combination therapy treatments, the sequence of drug application can impact the anti-proliferative effect of both compounds (19). Therefore, we examined the ability of AMD3100 pre-treatment to sensitize cells to chemotherapy with Taxol. Either ID8 or TOV-112D cells were first incubated in the presence or absence of AMD3100. Following pre-treatment, cells were collected, viability determined, and equal numbers of viable cells were redistributed. The re-plated cells were incubated in media containing AMD3100, TAXOL, or both. In ID8 cells, untreated cells re-plated in AMD3100 media proliferated 22% more than untreated cells, though not significantly so. In contrast, proliferation of ID8 cells pre-treated with AMD3100 and reseeded in AMD-3100 media was reduced by 48%, as compared to untreated control cells (P=0.0050) (FIG. 4A). ID8 cells re-plated in TAXOL (0.005 μM) without AMD3100 pre-treatment reduced cell proliferation by 50% (P=0.0025), while AMD3100 pre-treatment followed by TAXOL reduced cell growth by 60% compared to untreated cells (P=0.0002) (FIG. 4A). Pre-treatment of ID8 cells with AMD3100 did not sensitize cells to TAXOL as compared to untreated control cells that were then incubated with TAXOL (FIG. 4A). Similarly, pre-treatment did not enhance the sensitivity of ID8 cells to incubation with AMD3100 and TAXOL simultaneously (FIG. 4A). The simultaneous delivery of AMD3100 and TAXOL had the largest impact on cell proliferation, regardless of pre-incubation in control media or AMD3100 (86% and 84% respectively) when compared to untreated cells (P value <0.001)(FIG. 4A).

The impact of pre-treatment with AMD3100 was determined for TOV-112D cells in a similar manner. Proliferation of TOV-112D cells incubated in control media and subsequently treated with AMD3100 or TAXOL were reduced 51% and 24% respectively (FIG. 4B). TOV-112D cells incubated first with AMD3100 and then re-plated in control media or AMD3100 for a further 96 hrs were also significantly reduced in their proliferation independent of the presence of TAXOL (49% and 79.5% respectively, P<0.0001), indicating that AMD3100 pre-treatment contributes significantly to inhibition of cell proliferation (FIG. 4B). Pre-treatment with AMD-3100 significantly sensitized TOV-112D cells, with subsequent treatment, TAXOL reduced proliferation by 50% and a combination of AMD-3100 and TAXOL resulted in an 87% reduction. Pre-treatment with AMD3100 followed by incubation with AMD3100 alone or the combination of AMD3100 and TAXOL was not significantly different, suggesting that continued incubation in AMD3100 drives the anti-proliferative effect. We found that TOV-112D cells were overall more sensitive to chemotherapy and AMD3100 treatment as compared to ID8 cells, requiring a lower dose of both drugs to not kill the majority of cells. For TOV-112D cells, AMD3100 pre-treatment enhances sensitivity to AMD3100 and though not low-dose TAXOL.

### Example 7

### AMD3100 and Low Dose TAXOL Limit ID8 and TOV-112D Colony Formation

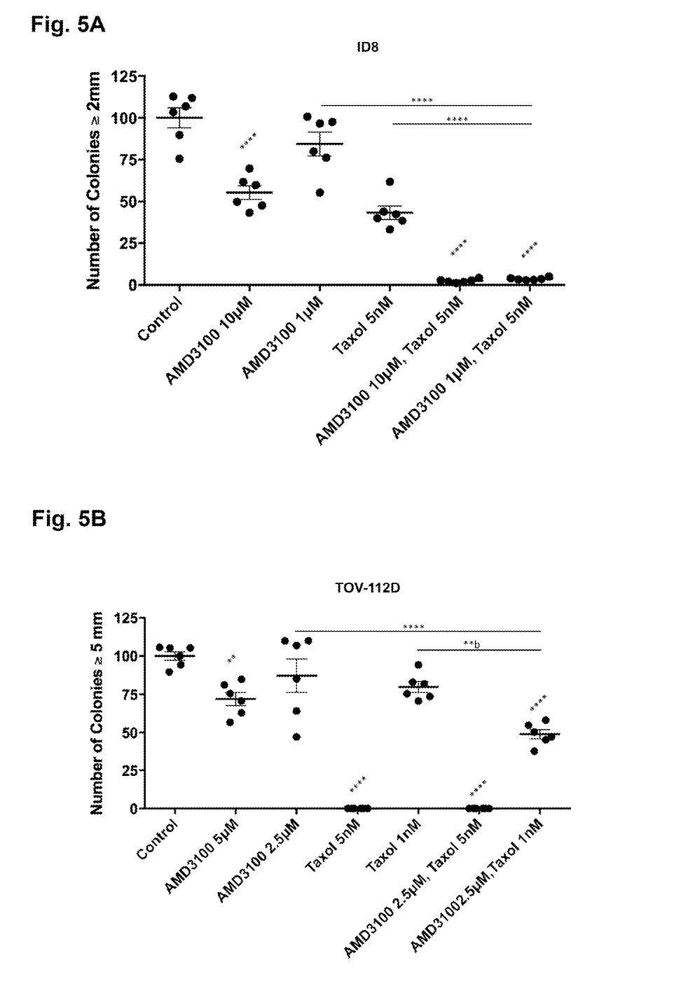

To further characterize the effect of AMD3100 and TAXOL treatment on cell proliferation, we conducted soft-agar colony formation assays with ID8 and TOV-112D cells. 1D8 cells treated with AMD3100 (10 and 1 M) decreased colony formation by 44% and 16% respectively. Incubation with TAXOL (15 nM and 5 nM) had a greater effect on ID8 colony formation and reduced ID8 colony formation by 94% and 56% respectively (FIG. 5A). Co-incubation with both AMD3100 and TAXOL reduced colony formation by 96% compared to untreated cells (P Value <0.0001) (FIG. 5A). We observed a significant reduction in cell growth with AMD3100 and TAXOL combination therapy compared to TAXOL treatment alone (P Value<0.0001). The difference was still significant when we combined 10 fold less concentrated AMD3100 (1 μM) with TAXOL (0.005 M) (P Value <0.0001).

In TOV-112D cells, AMD3100 at 2.5 μM did not reduce colony formation while TAXOL at 1 nM alone reduced colony formation only by 20% (non-significant P values for both). Co-incubation of cells with AMD3100 and TAXOL reduced colony formation by 50% (P Value <0.0001) (FIG. 4B). Together, these data demonstrate that AMD3100 and low-dose TAXOL delivered in combination limit the capacity of mouse and human ovarian cancer cells to form colonies on soft agar.

### Example 8

This study demonstrates that the combination of AMD3100 with low-dose paclitaxel improves the anti-proliferative effect of the chemotherapeutic agent as compared to either drug alone, supporting the further development of AMD3100 as an adjunctive therapy for chemotherapeutic treatment of ovarian cancer. Paclitaxel and cisplatin are the two chemotherapy drugs used as first line treatments for advanced stage ovarian cancers, especially Epithelial Ovarian Cancer (EOC) (29). Paclitaxel stabilizes microtubules, leading to mitosis arrest and cell death (30). Unfortunately, paclitaxel therapy can lead to several life-threatening complications such as generalized urticaria, angioedema, bronchospasm, and hypotension. Thus, many patients with ovarian cancer must forego or abbreviate paclitaxel therapy due to toxicity issues (31). Standard clinical doses of chemotherapeutic agents can exhibit significant side effects, there is a growing interest to use molecular-targeting therapies in combination with low dose chemotherapeutic agents with the aim to reduce toxicity and preserve and/or improve the efficacy of anti-cancer treatments. Proadifen, cucurbitacin B, and gold nanoparticles are among those agents shown to sensitize ovarian cancer cells to either cisplatin or paclitaxel (TAXOL) (6,27,28). The IC₅₀ of paclitaxel in human ovarian cancer is reported between ≤0.1 nM to 3 μM (32-35).

We found that for ID8 murine ovarian cancer cells the IC₅₀ of TAXOL was 5 nM for and 2.5 nM for human ovarian cancer cells (TOV). Treatment with AMD3100 in combination with TAXOL significantly reduced the proliferation of both human and mouse ovarian cancer cells with as compared to TAXOL alone. To our knowledge, this study is the first to demonstrate the capacity of AMD3100 to enhance the sensitivity of ovarian cancer cells to reduced concentrations of TAXOL in-vitro. Our data demonstrate that TOV-112D human ovarian cancer cells exhibit increased sensitivity to low dose TAXOL (5 nM) (89% proliferation inhibition) as compared to 1D8 murine ovarian cancer cells (50% proliferation inhibition). Similarly, colony formation by TOV-112D cells also exhibited increased sensitivity to TAXOL as compared to ID8 cells: 5 nM TAXOL reduced ID8 colony formation by 57% and while completely preventing TOV-112D colony formation. These data suggest that human ovarian cancer may be more amenable to combination therapy with AMD3100.

We explored the capacity of pre-treatment with AMD3100 to sensitize ovarian cancer cells to low-dose paclitaxel (TAXOL). Consecutive treatments with AMD3100 significantly inhibited ID8 cell proliferation (48%) as compared to those cells treated with AMD3100 and then incubated without drug, which increased growth (22%) (FIG. 4A). The anti-proliferative effect was more prominent when TAXOL was combined with the second dose of AMD3100 and reduced the cell growth by 85%. Similar results were obtained with TOV-112D cells, which exhibited a more pronounced effect of simultaneous drug administration following AMD3100 pretreatment. These anti-proliferative results required the simultaneous presence of AMD3100 and TAXOL. Pre-treatment with AMD3100 did not significantly sensitize ID8 or TOV-112D to subsequent incubation with TAXOL alone. This finding contrasts with recent studies that demonstrate the sequence of drug treatment can increase the efficacy of combination therapies, though this may reflect distinctions in the mechanism of action targeted by each combination (19,36). Kim et al. showed that AMD3100 has a biphasic effect on proliferation of Myeloma cells. After incubating cells with AMD3100 for up to 14 days, the investigators concluded that AMD3100 enhances the proliferation of Multiple Myeloma cells initially then slows it down and subsequently induces a rapid cell death (37). AMD-3100 is also reported to be an agonist for CXCR7 (38).

AMD3100 was originally developed to inhibit the entry of CXCR4 tropic human immunodeficiency virus (HIV) into human CD4+ T cells. It was later found that AMD3100 mobilizes leukocytes from their primary immune cites to the blood, secondary organs, and peripheral tissues (39, 40,41). Oncologists have taken advantage of this activity of AMD3100 with the aim to improve the efficacy of current treatments for the hematologic cancers such as Multiple Myeloma and Non-Hodgkin's Lymphoma (42,43). Righi et al. demonstrated that AMD3100 can favorably alter the CD8+/T-reg ratio in a murine ovarian cancer model (10). The potential utility of CXCR4 inhibitors has been investigated in several cancers including B-cell lymphoma (20), glioblastoma (21), breast (22), colon (23), lung (24), gastric (25), and thyroid cancers (26). In preclinical studies in ovarian cancer, AMD3100 has been shown to reduce tumor dissemination in vivo (10,18).

### Example 9

### Subtherapeutic Dosing of AMD3100 in Murine Ovarian Cancer (ID8) and Mesothelioma (40L)

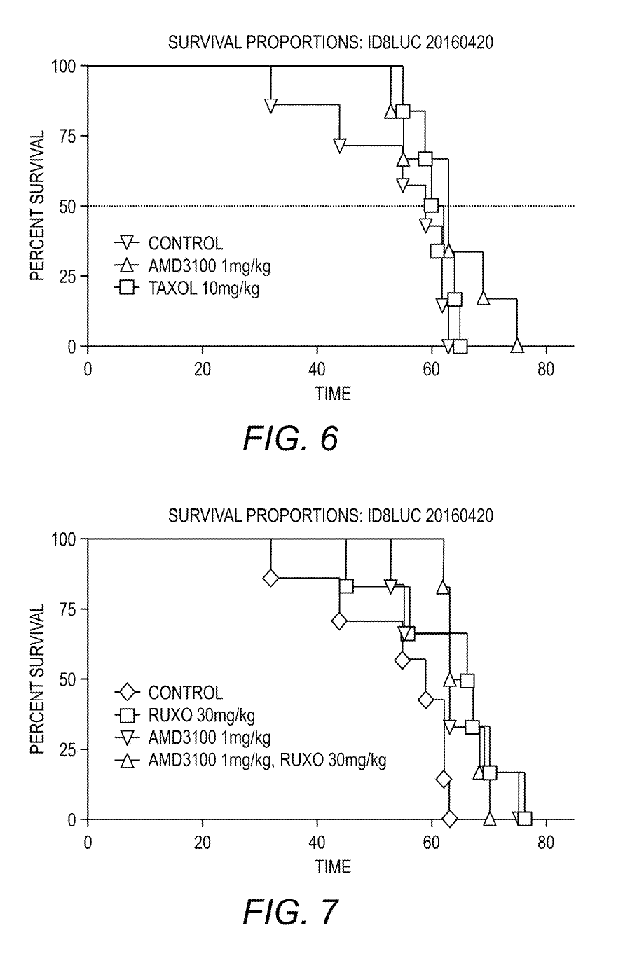

Six- to eight-week old C57/B6 mice were injected with 5e6 ID8-Luc cells. The presence of the tumor was confirmed by luciferase imaging. The tumor bearing mice were treated with 1 mg/kg AMD-3100 by intraperitoneal (IP) injection every 72 hours or with TAXOL® (10 mg/ml). The results are shown in FIG. 6. Subtherapeutic doses of ADM3100 extends the survival of the tumor bearing mice.

Six- to eight-week old C57/B16 mice were injected with 5e6 ID8-Luc cells. The presence of the tumor was confirmed by luciferase imaging. The tumor bearing mice were treated with 1 mg/kg AMD-3100 by IP injection every 72 hours. In the combination treatment, the AMD3100 (1 mg/kg) and ruxolitinib (30 mg/kg) were administered concurrently but not simultaneously (i.e., administration was staggered). The results are shown in FIG. 7. Subtherapeutic doses of ADM3100 extends the survival of the tumor bearing mice.

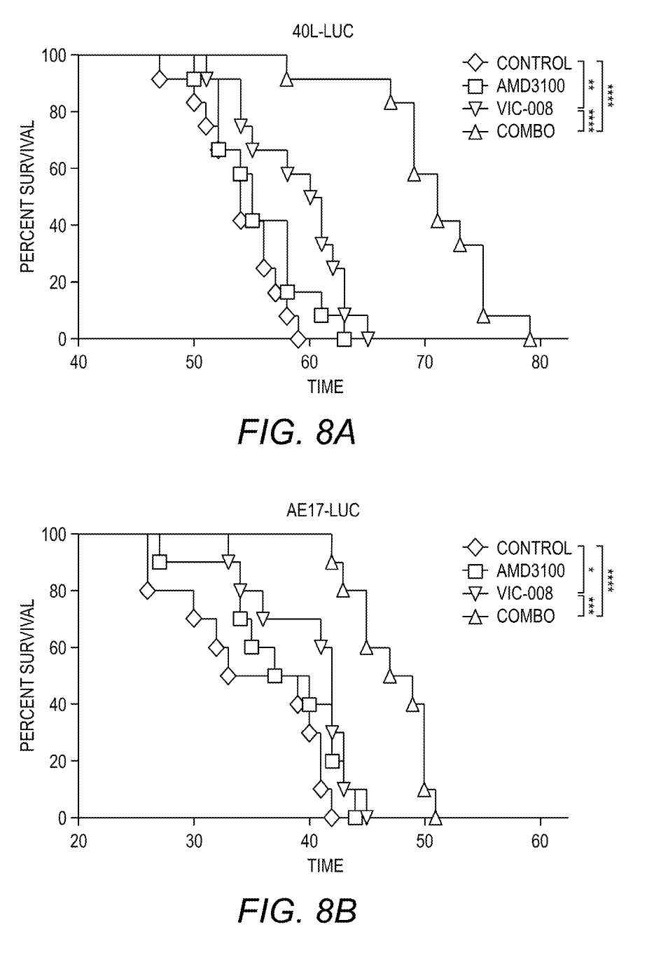

Intraperitoneal malignant mesothelioma (MM) models were established in immunocompetent C57BL/6 mice using syngeneic 40L and AE17 cell lines. Here, we tested the effect of AMD3100 and VIC-008, alone or in combination, on mouse survival in the mesothelioma-bearing mice. VIC-800, alone, was administered at 20 μg per mouse intraperitoneally (IP) once a week for four successive weeks. AMD3100 was administered at 1 mg/kg of mouse body weight via IP injection once a week for four successive weeks. In the combination treatment, the AMD3100 and VIC-008 were administered concurrently but not simultaneously (i.e., administration was staggered). As shown in FIGS. 8A-8B, VIC-008 alone prolonged animal survival in both 40L and AE17 models compared to saline control. AMD3100 alone seemed to confer modest benefit to survival in both 40L and AE17 mouse MM models compared to saline control (FIGS. 8A-8B). The combination treatment with VIC-008 and AMD3100 significantly prolonged animal survival compared to saline control in both 40L and AE17 models (FIGS. 8A-8B). Moreover, the combination treatment further significantly prolonged animal survival compared to VIC-008 treatment alone (FIGS. 8A-8B).

These studies showed that Subtherapeutic dosing of AMD3100 alone extends the survival of tumor bearing mice in ID8 and 40L tumor models alone and in combination with VIC008, respectively. Standard allopathic dose of AMD3100 in mice is 3 mg/kg/d. The therapeutic dose of AMD3100 used in these studies was 1 mg/kg/q72 h.

### REFERENCES

1. Siegel R L, Miller K D, Jemal A. Cancer statistics. C A Cancer J Clin. 2016; 66:7-30.
2. Jelovac D, Armstrong D K. Recent progress in the diagnosis and treatment of ovarian cancer. C A Cancer J Clin. 2011; 61:183-203.
3. Taïeb S, Ben Haj Amor M, Mailliez A, Adenis C, Leblanc É. D??pistage des cancers de l'ovaire: chez qui, comment et pour quelle efficacit?? ? Imag la Femme. 2016; 26:66-71.
4. Chan J K, Cheung M K, Husain A, Teng N N, West D, Whittemore A S, et al. Patterns and progress in ovarian cancer over 14 years. Obstet Gynecol. 2006; 108:521-8.
5. Siegel R L, Miller K D, Jemal A. Cancer statistics, 2016. CA Cancer J Clin. 2016; 66:7-30.
6. El-Senduny F F, Badria F A, E L-Waseef A M, Chauhan S C, Halaweish F. Approach for chemosensitization of cisplatin-resistant ovarian cancer by cucurbitacin B. Tumor Biol. 2016; 37:685-98.
7. Whicker M E, Lin Z P, Hanna R, Sartorelli A C, Ratner E S. MK-2206 sensitizes BRCA-deficient epithelial ovarian adenocarcinoma to cisplatin and olaparib. BMC Cancer. 2016; 16:550.
8. Katsman A, Umezawa K, Bonavida B. Chemosensitization and Immunosensitization of Resistant Cancer Cells to Apoptosis and Inhibition of Metastasis by the Specific NF-&#954; B Inhibitor DHMEQ. Curr Pharm Des. 2009; 15:792-808.
9. Scotton C J, Wilson J L, Milliken D, Stamp G, Balkwill F R. Epithelial cancer cell migration: A role for chemokine receptors? Cancer Res. 2001; 61:4961-5.
10. Righi E, Kashiwagi S, Yuan J, Santosuosso M, Leblanc P, Ingraham R, et al. CXCL12/CXCR4 blockade induces multimodal antitumor effects that prolong survival in an immunocompetent mouse model of ovarian cancer. Cancer Res. 2011; 71:5522-34.
11. Barbolina M V, Kim M, Liu Y, Shepard J, Belmadani A, Miller R J, et al. Microenvironmental regulation of chemokine (C—X—C-motif) receptor 4 in ovarian carcinoma. Mol Cancer Res. 2010; 8:653-64.
12. Rajagopal S, Kim J, Ahn S, Craig S, Lam C M, Gerard N P, et al. Beta-arrestin—but not G protein-mediated signaling by the “decoy” receptor CXCR7. Proc Natl Acad Sci USA. 2010; 107:628-32.
13. Circelli L, Sciammarella C, Guadagno E, Tafuto S, del Basso de Caro M, Botti G, et al. CXCR4/CXCL12/CXCR7 axis is functional in neuroendocrine tumors and signals on mTOR. Oncotarget. 2016; 7:18865-75.
14. Xu D, Li R, Wu J, Jiang L, Zhong H A. Drug Design Targeting the CXCR4/CXCR7/CXCL12 Pathway. Curr Top Med Chem. 2016; 16:1441-51.
15. Mcconnell A T, Ellis R, Pathy B, Plummer R, Lovat P E, O'Boyle G. The prognostic significance and impact of the CXCR4-CXCR7-CXCL12 axis in primary cutaneous melanoma. Br J Dermatol. 2016; 1210-20.
16. Fu X, Tao L, Rivera A, Williamson S, Song X-T, Ahmed N, et al. A simple and sensitive method for measuring tumor-specific T cell cytotoxicity. PLoS One. 2010; 5:e11867.
17. Zhu X, Bai Q, Lu Y, Lu Y, Zhu L, Zhou X, et al. Expression and function of CXCL12/CXCR4/CXCR7 in thyroid cancer. Int J Oncol. 2016; 48:2321-9.
18. Kajiyama H, Shibata K, Terauchi M, Ino K, Nawa A, Kikkawa F. Involvement of SDF-1??/CXCR4 axis in the enhanced peritoneal metastasis of epithelial ovarian carcinoma. Int J Cancer. 2008; 122:91-9.
19. Lee M J, Ye A S, Gardino A K, Heijink A M, Sorger P K, MacBeath G, et al. Sequential Application of Anticancer Drugs Enhances Cell Death by Rewiring Apoptotic Signaling Networks. Cell. 2012; 149:780-94.
20. Reinholdt L, Laursen M B, Schmitz A, Bedker J S, Jakobsen L H, Begsted M, et al. The CXCR4 antagonist plerixafor enhances the effect of rituximab in diffuse large B-cell lymphoma cell lines. Biomark Res. Biomarker Research; 2016; 4:12.
21. Weder N, Zhang H, Jensen K, Yang B Z, Simen A, Jackowski A, et al. Child abuse, depression, and methylation in genes involved with stress, neural plasticity, and brain circuitry. J Am Acad Child Adolesc Psychiatry. 2014; 53:417-24.e5.
22. Lefort S, Thuleau A, Kieffer Y, Sirven P, Bieche I, Marangoni E, et al. CXCR4 inhibitors could benefit to HER2 but not to triple-negative breast cancer patients. Oncogene. Nature Publishing Group; 2016; 1-12.
23. Mustafi R, Dougherty U, Mustafi D, Ayaloglu-Butun F, Fletcher M, Adhikari S, et al. ADAM 17 is a tumor promoter and therapeutic target in Western diet-associated colon cancer. Clin Cancer Res. 2016; clincanres.3140.2016.
24. Singla A K, Downey C M, Bebb G D, Jirik F R. Characterization of a murine model of metastatic human non-small cell lung cancer and effect of CXCR4 inhibition on the growth of metastases. Oncoscience. 2015; 2:263-71.
25. Qiao J, Liu Z, Yang C, Gu L, Deng D. SRF promotes gastric cancer metastasis through stromal fibroblasts in an SDF1-CXCR4-dependent manner. Oncotarget. 2014; 7.
26. Stoor P, Pulkkinen J, Grenman R. Bioactive glass S53P4 in the filling of cavities in the mastoid cell area in surgery for chronic otitis media. Ann Otol Rhinol Laryngol. 2010; 119:377-82.
27. Jendželovský R, Jendželovská Z, Hil'ovska L, Kovarl'J, Mikeš J, Fedoročko P. Proadifen sensitizes resistant ovarian adenocarcinoma cells to cisplatin. Toxicol Lett. 2016; 243:56-66.
28. Xiong X, Arvizo R R, Saha S, Robertson D J, McMeekin S, Bhattacharya R, et al. Sensitization of ovarian cancer cells to cisplatin by gold nanoparticles. Oncotarget. 2014; 5:6453-65.
29. Townsley C, Oza A. Antiangiogenic therapies in ovarian cancer. Therapy. 2010; 7:277-84.
30. Tsukada T, Fushida S, Harada S, Terai S, Yagi Y, Kinoshita J, et al. Low-dose paclitaxel modulates tumour fibrosis in gastric cancer. Int J Oncol. 2013; 42:1167-74.
31. Yanaranop M, Chaithongwongwatthana S. Intravenous versus oral dexamethasone for prophylaxis of paclitaxel-associated hypersensitivity reaction in patients with primary ovarian, fallopian tube and peritoneal cancer: A double-blind randomized controlled trial. Asia Pac J Clin Oncol. 2016; 12:289-99.
32. Pal M K, Jaiswar S P, Srivastav A K, Goyal S, Dwivedi A, Verma A, et al. Synergistic effect of piperine and paclitaxel on cell fate via cyt-c, Bax/Bcl-2-caspase-3 pathway in ovarian adenocarcinomas SKOV-3 cells. Eur J Pharmacol. Elsevier; 2016; 791:751-62.
33. Li S, Yang L, Wang J, Liang F, Chang B, Gu H, et al. Analysis of the chemotherapeutic effects of a propadiene compound on malignant ovarian cancer cells. Oncotarget. 2016; 7.
34. Puvanenthiran S, Essapen S, Seddon A, Modjtahedi H. Impact of the putative cancer stem cell markers and growth factor receptor expression on the sensitivity of ovarian cancer cells to treatment with various forms of small molecule tyrosine kinase inhibitors and cytotoxic drugs. Int J Oncol. 2016; 1-14.
35. Zhou J, Alfraidi A. Zhang S, Santiago-O'Farrill J, Yerramreddy V, Alsaadi A, et al. A novel compound ARN-3236 inhibits Salt Inducible Kinase 2 and sensitizes ovarian cancer cell lines and xenografts to paclitaxel. Clin Cancer Res. 2016; 1-10.
36. Zhang R, Yang J, Sima M, Zhou Y, Kopeček J. Sequential combination therapy of ovarian cancer with degradable N-(2-hydroxypropyl)methacrylamide copolymer paclitaxel and gemcitabine conjugates. Proc Natl Acad Sci USA. 2014; 111:12181-6.
37. Kim H-Y, Hwang J-Y, Kim S-W, Lee H-J, Yun H-J, Kim S, et al. The CXCR4 Antagonist AMD3100 Has Dual Effects on Survival and Proliferation of Myeloma Cells In Vitro. Cancer Res Treat. 2010; 42:225.
38. Kalatskaya I, Berchiche Y A, Limberg B J, Rosenbaum J S, Heveker N. AMD3100 Is a CXCR7 Ligand with Allosteric Agonist Properties. 2009; 75:1240-7.
39. Kean L S, Sen S, Onabajo O, Singh K, Robertson J, Stempora L, et al. Significant mobilization of both conventional and regulatory T cells with AMD3100. Blood. 2011; 118:6580-90.
40. Broxmeyer H E, Orschell C M, Clapp D W, Hangoc G, Cooper S, Plett P A, et al. Rapid mobilization of murine and human hematopoietic stem and progenitor cells with AMD3100, a CXCR4 antagonist. J Exp Med. 2005; 201:1307-18.
41. Pusic I, DiPersio J F. Update on clinical experience with AMD3100, an SDF-1/CXCL12-CXCR4 inhibitor, in mobilization of hematopoietic stem and progenitor cells. Curr Opin Hematol. 2010; 17:319-26.
42. Costa L J, Abbas J, Hogan K R, Kramer C, McDonald K, Butcher C D, et al. Growth factor plus preemptive (‘just-in-time’) plerixafor successfully mobilizes hematopoietic stem cells in multiple myeloma patients despite prior lenalidomide exposure. Bone Marrow Transplant. Nature Publishing Group; 2012; 47:1403-8.
43. Wagstaff A J. Plerixafor: in patients with non-Hodgkin's lymphoma or multiple myeloma. Drugs. 2009; 69:319-26.

The foregoing is illustrative of the present invention, and is not to be construed as limiting thereof. The invention is defined by the following claims, with equivalents of the claims to be included therein.

1. A method for killing a cancer cell expressing an amount of a chemokine sufficient to produce a chemorepellent effect in a subject in need thereof, which method comprises:
a) contacting the cancer cell with an amount of a CXCL12 signaling inhibitor for a sufficient period of time to inhibit the chemorepellent effect and to increase migration of immune cells to the cancer cell;
b) contacting the cancer cell with a subtherapeutic amount of a chemotherapeutic agent, and
c) optionally repeating steps a) and b) as necessary to kill the cancer cell.

2. The method of claim 1, wherein the cancer cell is periodically contacted with the CXCL12 signaling inhibitor.

3. The method of claim 1, wherein the cancer cell is in a solid tumor.

4. (canceled)

5. A method for enhancing the therapeutic effect of a chemotherapeutic agent on a tumor in a subject, the tumor expressing an amount of a chemokine sufficient to produce a chemorepellent effect, which method comprises:
a) selecting a subject having a tumor expressing an amount of a chemokine sufficient to produce a chemorepellent effect,
b) administering an effective amount of a CXCL12 signaling inhibitor to the subject having the tumor for a sufficient time to increase penetration of immune cells into the tumor; and
c) administering a subtherapeutic amount of the chemotherapeutic agent to the subject,
wherein the therapeutic effect of the subtherapeutic amount of the chemotherapeutic on the tumor is enhanced as compared to the subtherapeutic amount administered without the CXCL12 signaling inhibitor.

6. A method for increasing immune cell migration into a tumor, which method comprises
a) identifying a tumor having a chemorepellent property whereby immune cells are repelled from the tumor,
b) contacting the tumor with an amount of a CXCL12 signaling inhibitor for a sufficient period of time to inhibit the chemorepellent effect;
c) contacting the tumor with a subtherapeutic amount of a chemotherapeutic agent, and
d) optionally repeating steps b) and c),
whereupon the migration of immune cells into the tumor is increased.

7. The method of claim 6, wherein the tumor is periodically contacted with the CXCL12 signaling inhibitor.

8. The method of claim 1, wherein the CXCL12 signaling inhibitor and the chemotherapeutic agent are administered concurrently.

9. The method of claim 1, wherein the CXCL12 signaling inhibitor and the chemotherapeutic agent are administered sequentially.

10. The method of claim 1, wherein the CXCL12 signaling inhibitor is administered up to 3 days before administering the chemotherapeutic agent.

11. The method of claim 1, wherein the CXCL12 signaling inhibitor is administered before administering the chemotherapeutic agent.

12. The method of claim 1, wherein the CXCL12 signaling inhibitor is selected from the group consisting of AMD3100, AMD11070, AMD12118, AMD 11814, AMD13073, FAMD3465, C131, BKT140, CTCE-9908, KRH-1636, KRH-2731, KRH-3955, TC14012, BMS-936564/MDX-1338, LY2510924, GSK812397, T-20, T-22, T-140, TE-14011, T-14012, TN14003, TAK-779, AK602, SCH-351125, Tannic acid, NSC 651016, thalidomide, and GF 109230X.

13. The method of claim 1, wherein the chemokine is CXCL12 or interleukin 8.

14. The method of claim 1, wherein the cancer cell or the tumor is an ovarian cancer cell or ovarian cancer.

15. The method of claim 1, wherein the cancer cell or the tumor is an epithelial ovarian cancer cell or epithelial ovarian cancer.

16. The method of claim 1, wherein the cancer cell or the tumor is an ovarian cancer cell or ovarian cancer that has progressed to recurrent platinum resistant disease.

17. The method of claim 1, wherein the subject has a recurrence of epithelial ovarian cancer, primary peritoneal cancer, or fallopian tube cancer.

18. The method of claim 1, wherein the chemotherapeutic agent is a taxane.

19. The method of claim 1, wherein the chemotherapeutic agent is paclitaxel.

20. (canceled)

21. The method of claim 1, wherein the chemotherapeutic agent is albumin-bound paclitaxel.

22-24. (canceled)
